(19) United States
(12) Patent Application Publication
(10) Pub. No.: US 2021/0061876 A1
(43) Pub. Date: Mar. 4, 2021

(54) T CELL RECEPTORS FOR TUMOR SPECIFIC PROTEASOME SPLICE VARIANTS AND USES THEREOF

(71) Applicants: Max-Delbrück-Centrum für Molekulare Medizin in der Helmholtz-Gemeinschaft, Berlin (DE); Deutsches Krebsforschungszentrum Stiftung des öffentlichen Rechts, Heidelberg (DE); Imperial College Innovations Limited, London (GB); Charité - Universitätsmedizin Berlin, Berlin (DE)

(72) Inventors: Thomas BLANKENSTEIN, Berlin (DE); Gerald WILLIMSKY, Berlin (DE); Juliane LIEPE, Göttingen (DE); Peter Michael KLOETZEL, Berlin (DE); Michele MISHTO, London (GB); Christin BEIER, Berlin (DE)

(73) Assignees: Max-Delbrück-Centrum für Molekulare Medizin in der Helmholtz-Gemeinschaft, Berlin (DE); Deutsches Krebsforschungszentrum Stiftung des öffentlichen Rechts, Heidelberg (DE); Imperial College Innovations Limited, London (GB); Charité - Universitätsmedizin Berlin, Berlin (DE)

(21) Appl. No.: 16/958,604

(22) PCT Filed: Jan. 2, 2019
(86) PCT No.: PCT/EP2019/050027

§ 371 Date: Jun. 26, 2020

(30) Foreign Application Priority Data
Dec. 29, 2017 (EP) 17211094.2

(51) Int. Cl. C07K 14/725; A61K 38/00; A61K 39/00

(52) U.S. Cl. CPC C07K 14/7051; A61K 39/00; A61K 38/00

(57) ABSTRACT

The present invention pertains to antigen recognizing constructs against tumor specific proteasome splicing variants. The invention in particular provides novel T cell receptor (TCR) based molecules which are selective and specific for tumor cells carrying antigenic epitopes generated by proteasome peptide splicing of tumor specific antigens. The TCRs of the invention, and antigen binding fragments derived therefrom, are of use for the diagnosis, treatment and prevention of proliferative diseases, preferably for the treatment of cancer. Further provided are nucleic acids encoding the antigen recognizing constructs of the invention, vectors comprising these nucleic acids, recombinant cells expressing the antigen recognizing constructs and pharmaceutical compositions comprising the compounds of the invention.

### FIELD OF THE INVENTION

The present invention pertains to antigen recognizing constructs against tumor specific proteasome splicing variants. The invention in particular provides novel T cell receptor (TCR) based molecules which are selective and specific for tumor cells carrying antigenic epitopes generated by proteasome peptide splicing of tumor specific antigens. The TCR of the invention, and antigen binding fragments derived therefrom, are of use for the diagnosis, treatment and prevention of proliferative diseases, preferably for the treatment of cancer. Further provided are nucleic acids encoding the antigen recognizing constructs of the invention, vectors comprising these nucleic acids, recombinant cells expressing the antigen recognizing constructs and pharmaceutical compositions comprising the compounds of the invention.

### DESCRIPTION

A number of human cancers have been shown to be associated with characteristic mutations in genes governing the production of proteins involved in cell division. The ras oncogene and its Ras protein gene product are mutated in many solid tumors. It is estimated that ras mutations are found in approximately 180,000 new cancer cases each year in the United States across a spectrum of tumor types, including pancreas, non-small cell lung cancers (NSCLC), colorectal, endometrial and ovarian cancers, melanoma and multiple myeloma. Ras-mutated pancreas cancer has a particularly poor prognosis. The American Cancer Society predicted that in the United States in 2013 there would be 45,220 new cases of pancreas cancer diagnosed and 38,460 deaths from pancreas cancer. Therapies targeting Ras mutation positive cancers are therefore urgently needed.

The 20S proteasome particle with its active site β-subunits β1, β2 and β5 is a N-terminal nucleophilic hydrolase. It is the central catalytic unit of the ubiquitin proteasome system (UPS) and the catalytic core of the 26S proteasome that is built by the association of the two 19S regulator complexes with the catalytic 20S proteasome particle core. As part of the 26S proteasome, the 20S proteasome particle degrades poly-ubiquitinated proteins to peptides of 3 to 20 residues in length (Schwartz AL, Ciechanover A (2009) Targeting proteins for destruction by the ubiquitin system: implications for human pathobiology. Annu Rev Pharmacol Toxicol 49: 73-96). IFN-γ induces the synthesis of alternative catalytic subunits and the concomitant formation of immunoproteasomes previously connected the UPS with the generation of virus or tumor derived antigenic peptides bound by MHC class I molecules and presented at the cell surface to CD8+cytotoxic T lymphocytes (CTL) for immune recognition (Kloetzel P M (2001) Antigen processing by the proteasome. Nat Rev Mol Cell Biol 2: 179-187).

Until recently it appeared to be a canonical rule that peptides generated by the 20S proteasome particle are peptide fragments with a sequence identical to a linear contiguous sequence part present in the unprocessed parental protein. However, using patient derived CTLs and breaking this rule, two new epitope peptides were recently identified which were composed of two different peptide fragments of a parental protein and whose sequence was not identical to a linear sequence part present in the parent protein. These peptides were the result of a new mechanism called peptide-splicing and it was shown that proteasomes generated these spliced epitope peptides both in vivo and in vitro (Vigneron N, Stroobant V, Chapiro J, Ooms A, Degiovanni G, et al. (2004) An antigenic peptide produced by peptide splicing in the proteasome. Science 304: 587-590; and Warren E H, Vigneron N J, Gavin M A, Coulie P G, Stroobant V, et al. (2006) An antigen produced by splicing of noncontiguous peptides in the reverse order. Science 313: 1444-1447). Proteasome catalyzed peptide splicing was proposed to be a transpeptidation reaction whereby the acylester intermediate is stabilized at the active site formed by the N-terminal threonine of the catalytic subunits for a time span that is sufficient to allow the N-termini of released peptide fragments to make a nucleophilic attack on the ester bond of the acyl-enzyme intermediate thereby forming a new peptide bond and producing the spliced peptides (Borissenko L, Groll M (2007) Diversity of proteasomal missions: fine tuning of the immune response. Biol Chem 388: 947-955). Furthermore, it was shown that two non-contiguous peptides can also be fused by splicing in a reversed order.

T-cell based immunotherapy targets represent peptide epitopes derived from tumor-associated or tumor-specific proteins, which are presented by molecules of the major histo-compatibility complex (MHC). These tumor associated antigens (TAAs) or tumor specific antigens (TSA) can be peptides derived from all protein classes, such as enzymes, receptors, transcription factors, etc. which are expressed and, as compared to unaltered cells of the same origin, usually up-regulated in cells of the respective tumor. In case the tumor is associated with a viral infection, such as with Merkel cell virus (MCV) or Human papilloma viruses (HPVs), immune therapy can be developed based on the virus antigens expressed by tumor cells which originate from virus infected host cells.

Specific elements of the cellular immune response are capable of specifically recognizing and destroying tumor cells. The isolation of T-cells from tumor-infiltrating cell populations or from peripheral blood suggests that such cells play an important role in natural immune defense against cancer. CD8-positive T-cells in particular, which recognize class I molecules of the major histocompatibility complex (MHC)-bearing peptides of usually 8 to 10 amino acid residues derived from proteins or defective ribosomal products (DRiPs) located in the cytosol, play an important role in this response. The MHC-molecules of the human are also designated as human leukocyte-antigens (HLA).

There are two classes of MHC-molecules, MHC class I and MHC class II. Complexes of peptide and MHC class I are recognized by CD8-positive T-cells bearing the appropriate T-cell receptor (TCR), whereas complexes of peptide and MHC class II molecules are recognized by CD4-positive-helper-T-cells bearing the appropriate TCR. Since both types of response, CD8 and CD4 dependent, contribute jointly and synergistically to the anti-tumor effect, the identification and characterization of tumor-associated and tumor-specific antigens and corresponding T cell receptors is important in the development of cancer immunotherapies such as vaccines and cell therapies.

In the MHC class I dependent immune reaction, peptides not only have to be able to bind to certain MHC class I molecules expressed by tumor cells, they subsequently also have to be recognized by T-cells bearing specific T-cell receptors (TCR). Therefore, TAAs and TSAs are a starting point for the development of a T-cell based therapy including but not limited to tumor vaccines and cell therapies.

A TCR is a heterodimeric cell surface protein of the immunoglobulin super-family, which is associated with invariant proteins of the CD3 complex involved in mediating signal transduction. TCRs exist in αβ and γδ forms, which are structurally similar but have quite distinct anatomical locations and probably functions. The extracellular portion of native heterodimeric αβTCR consists of two polypeptides, each of which has a membrane-proximal constant domain, and a membrane-distal variable domain. Each of the constant and variable domains includes an intra-chain disulfide bond. The variable domains contain the highly polymorphic loops analogous to the complementarity determining regions (CDRs) of antibodies. The use of TCR gene therapy overcomes a number of current hurdles. It allows equipping patients' own T cells with desired specificities and generation of sufficient numbers of T cells in a short period of time, avoiding their immediate exhaustion. The TCR will be transduced into central memory T cells or T cells with stem cell characteristics, which may ensure better persistence and function upon transfer. TCR-engineered T cells will be infused into cancer patients renderedlymphopenic by chemotherapy or irradiation, allowing efficient engraftment but inhibiting immune suppression.

While advances have been made in the development of molecular-targeting drugs for cancer therapy, there remains a need in the art to develop new anti-cancer agents that specifically target molecules highly specific to cancer cells. The present description addresses that need by providing novel TCRs directed to antigens out of the class of mutated tumor specific antigens (TSA) which are generated by peptide splicing in the proteasome. Such antigenic peptides do therefore not correspond to a linear peptide epitope found in the parent mutated antigenic protein. In context of the present invention spliced peptide variants of the mutated Ras^{G12V} antigen were identified and used for the generation of human T cell receptor constructs. Most preferably the TSA of the invention is therefore a peptide identified herein which are shown in table 2 below.

Immunogenic Spliced Peptides:

In one aspect of the invention there is provided a spliced ras (also referred to as “Kras”) antigen peptide epitope comprising a sequence according to a sequence shown in any one of SEQ ID NO: 76 to 78, and 163. The peptide of the invention may have a length of at least 9 amino acids, preferably 9 to 500, more preferably 9 to 50, 9 to 20, most preferably 9 to 12, or 9 to 11, or 9 to 10 amino acids. Preferably the peptide of the invention consists essentially of, or consists of, a peptide sequence shown in any of SEQ ID NO: 76 to 78, and 163. The peptide of the invention may be additionally provided as a retro-invers peptide or fusion peptide, potentially together with other TAA or TSA peptides.

Ras proteins bind GDP/GTP and possess intrinsic GTPase activity. In context of the herein disclosed invention the terms “ras” or “Kras” refer to the huma Kras protein which protein sequence can be derived from UniProt with the accession number Po1116 (www.uniprot.com, in the database version of December 2018). Ras plays an important role in the regulation of cell proliferation (PubMed:23698361, PubMed:22711838) and has a role in promoting oncogenic events by inducing transcriptional silencing of tumor suppressor genes (TSGs) in colorectal cancer (CRC) cells in a ZNF304-dependent manner (PubMed:24623306). The gene encoding for Kras protein is derivable from the www.genenames.org database with the accession number HGNC:6407, in the database version of December 2018. Both the protein and the gene sequence, as well as mRNA, are incorporated herein by reference to the abovementioned databases.

The spliced ras antigen peptide epitope of the invention is preferably an immunogenic peptide. In particular the peptide of the invention, when being in complex with an MHC, for example presented on an antigen presenting cell such as a dendritic cell, will induce an HLA restricted immune response, preferably via binding of the peptide to a T cell receptor. Preferably the peptides of the invention are HLA restricted peptides, most preferably the peptides are HLA A02 restricted peptides.

The immunogenic peptides of the invention are preferably of a sequence that is not present in a naturally occurring Kras protein or mutated Kras protein. In this regard the term “mutated Kras protein” shall refer to known Kras mutations that are present in the human population and often are associated with a disease such as cancer. Mutated Kras proteins are compared to the wild type human Kras sequence often mutated at position 12, and in preferred embodiments mutated Kras proteins shall refer to Kras proteins with an alternate sequence at this position.

Thus, another aspect of the present invention relates to the use of the immunogenic peptides according to the present invention for the treatment of a proliferative disease selected from the group of a cancer associated with Kras protein expression, or mutated Kras protein expression. The latter is preferred. Such cancers are disclosed herein below.

The present invention furthermore relates to peptides according to the present invention that have the ability to bind to a molecule of the human major histocompatibility complex (MHC) class-1 or—in an elongated form, such as a length-variant—MHC class-II.

The present invention further relates to the peptides according to the present invention wherein said peptides (each) consist or consist essentially of an amino acid sequence according to SEQ ID NO: 76 to SEQ ID NO: 78 and SEQ ID NO: 163.

The present invention further relates to the peptides according to the present invention, wherein said peptide is modified and/or includes non-peptide bonds.

The present invention further relates to the peptides according to the present invention, wherein said peptide is part of a fusion protein, in particular fused to the N-terminal amino acids of the HLA-DR antigen-associated invariant chain (1i), or fused to (or into the sequence of) an antibody, such as, for example, an antibody that is specific for dendritic cells.

The present invention further relates to a nucleic acid, encoding the peptides according to the present invention. The present invention further relates to the nucleic acid according to the present invention that is DNA, cDNA, PNA, RNA or combinations thereof.

The present invention further relates to an expression vector capable of expressing and/or expressing a nucleic acid according to the present invention. A preferred expression vector according to the invention is based on a viral genome backbone, therefore, viral, such as lenti viral or other, vectors are preferably included. Other expression systems may be based on transposable elements, CRISPR/Cas9 approaches, RNA transfection, or any method suitable and known to the skilled artisan.

The present invention further relates to a peptide according to the present invention, a nucleic acid according to the present invention or an expression vector according to the present invention for use in the treatment of diseases and in medicine, in particular in the treatment of diseases including cancer and autoimmune/inflammatory/immune pathological diseases.

The present invention further relates to antibodies against the peptides according to the present invention or complexes of said peptides according to the present invention with MHC, and methods of making these.

The present invention further relates to T-cell receptors (TCRs), in particular soluble TCR (sTCRs) and cloned TCRs engineered into autologous or allogeneic T cells, and methods of making these, as well as NK cells or other cells bearing said TCR or cross-reacting with said TCRs. The antibodies and TCRs are additional embodiments of the immunotherapeutic use of the peptides according to the invention at hand. The present invention further relates to a host cell comprising a nucleic acid according to the present invention or an expression vector as described before. The present invention further relates to the host cell according to the present invention that is an antigen presenting cell, and preferably is a dendritic cell.

The present invention further relates to a method for producing a peptide according to the present invention, said method comprising culturing the host cell according to the present invention, and isolating the peptide from said host cell or its culture medium.

The present invention further relates to said method according to the present invention, wherein the antigen is loaded onto class I or II MHC molecules expressed on the surface of a suitable antigen-presenting cell or artificial antigen-presenting cell by contacting a sufficient amount of the antigen with an antigen-presenting cell.

The present invention further relates to the method according to the present invention, wherein the antigen-presenting cell comprises an expression vector capable of expressing or expressing said peptide containing SEQ ID No. 76 to SEQ ID No.: 78 and SEQ ID NO: 163, preferably containing SEQ ID No. 76, or a variant amino acid sequence.

The present invention further relates to activated T cells, produced by the method according to the present invention, wherein said T cell selectively recognizes a cell which expresses a polypeptide comprising an amino acid sequence according to the present invention.

The present invention further relates to a method of killing target cells in a patient which target cells aberrantly express a Kras or mutated Kras polypeptide, the method comprising administering to the patient an effective number of T cells as produced according to the present invention. The present invention further relates to the use of any peptide as described, the nucleic acid according to the present invention, the expression vector according to the present invention, the cell according to the present invention, the activated T lymphocyte, the T cell receptor or the antibody or other peptide- and/or peptide-MHC-binding molecules according to the present invention as a medicament or in the manufacture of a medicament. Preferably, the medicament is active against cancer. For example, the peptides of the invention may be formulated as a vaccine composition, optionally together with an adjuvant, for use in the treatment of a cancer disease.

Preferably, said medicament is for a cellular therapy, a vaccine or a protein based on a soluble TCR or antibody. Instead of classical antibodies the invention shall also comprise known antibody variants such as membrane bound chimeric antigen receptors (CAR).

The present invention further relates to a use according to the present invention, wherein said cancer cells are cells of a cancer disclosed herein below.

Antigen Recognizing Constructs

In some aspects the herein disclosed invention pertains to an antigen recognizing construct that specifically binds to a spliced protein peptide epitope, wherein spliced peptide epitope consists of a sequence that does not exist in a full-length human protein sequence, more preferably not in a full length Kras protein.

The object of the invention is also solved by an antigen recognizing construct, such as a T cell receptor (TCR), specific and/or selective for a mutant ras epitope, preferably a Ras mutation at position G12, for example G12D or G12V, and even more preferably for a proteasomal spliced peptide antigen derived from the ras oncogene product, preferably, wherein the antigen comprises preferably the Ras^{G12} mutation, such as the Ras^{G12}D or Ras^{G12V} mutation. Most preferably, the antigen recognizing construct of the invention binds specifically to the mutant ras antigen peptide epitope, preferably both to the linear as well as the spliced peptide variants, as described before. However, in preferred aspects and embodiments of the invention, the herein disclosed antigen recognizing constructs bind specifically and selectively to the spliced variants disclosed herein. Other than the G12D or G12V mutation, the variants G12A G12S and G12C are other preferred mutated Ras epitopes recognized by the herein disclosed antigen recognizing constructs. Hence in some embodiments the antigen recognizing construct in accordance with the invention is preferably recognizing an MHC presented spliced peptide antigen derived from mutated Ras with the mutation G12D, G12V, G12A, G12S and/or G12C.

In accordance with the invention, an herein disclosed antigenic peptide recognized by the antigen recognizing constructs is a peptide that is presented by an MHC, preferably HLA A*02, and which does not consist of an amino acid sequence that is i00% identical to the amino acid sequence of a parent Ras sequence having any one of the variations: G12D, G12V, G12A, G12S and G12C.

The object of the invention is solved in an additional aspect by an antigen recognizing construct comprising at least one complementary determining region (CDR) 3 having at least 50%, 60%, 70%, 80%, 90%, 95%, 98%, 99%, or preferably 100% sequence identity to an amino acid sequence selected from SEQ ID NOs. 3, 7, 11, 15, 19, 23, 27, 31, 35, 39, 43, 47, 51, 55, 59, 63, 67, and 71.

In other embodiments the invention pertains to an antigen recognizing construct comprising at least one complementary determining region (CDR) 3 having at least 50%, 60%, 70%, 80%, 90%, 95%, 98%, 99%, or preferably 100% sequence identity to an amino acid sequence selected from SEQ ID NOs. 85, 89, 93, 97, 101, 105, 109, 113, 117, 121, 125, 129, 133, 137, 141, 145, 149, 153, 157, and 161 (CDR3s of the chains of table 1a).

In the context of the invention for all mentioned sequences that are disclosed in the form that they are grouped as being sequence identical to a certain parent sequence, it shall be understood that in preferred embodiments, as an alternative, such sequences comprise compared to the parent sequence not more than one, not more than two, not more than three, amino acid substitutions, additions, deletions, modifications or inversions, if the parent sequence is a CDR, in particular CDR3 sequence. It is particularly preferred that such variations are located at the respective first and/or last amino acid positions of such CDR sequences. In this embodiment, such sequences are preferably which have not more than one amino acid mutation. With regard to sequences which are variable region sequences, it may be part of the invention that they contain compared to the parent not more than 10, 9, 8, 7, 6, 5, 4, 3, 2, or 1, amino acid substitutions, additions, deletions, modifications or inversions, with increasing preference.

Amino acid substitutions are preferably conservative substitutions which will not result in a significant loss of ARC avidity or specificity. Significant is at least everything that is more than 90%, 80% or preferably 50% loss compared to the parent sequence. For example, conservative amino acid exchanges may be included in the first two, preferably the first, position of the CDR1, CDR2 or CDR2 region. Other preferred positions are the last two, preferably the last, amino acid positions of the herein disclosed CDR1, CDR2, CDR3 sequences. As an example, the present invention shows that in SEQ ID NO:5, the first amino acid position may be exchanged without loss of avidity (M to L exchange).

The term “conservative amino acid substitutions” or similar terms, involves replacement of the aliphatic or hydrophobic amino acids Ala, Val, Leu and Ile; replacement of the hydroxyl residues Ser and Thr; replacement of the acidic residues Asp and Glu; replacement of the amide residues Asn and Gln, replacement of the basic residues Lys, Arg, and His; replacement of the aromatic residues Phe, Tyr, and Trp, and replacement of the small-sized amino acids Ala, Ser, Thr, Met, and Gly.

In some embodiments the antigen recognizing construct of the invention specifically binds to a TSA-peptide-HLA molecule complex, wherein the TSA peptide comprises, or alternatively consists of, a variant of the TSA which is at least 66%, preferably at least 77%, and more preferably at least 88% homologous (preferably at least 77% or at least 88% identical) to the amino acid sequence of the TSA of the invention (in particular SEQ ID NO 76 to 78, and 163), wherein said variant binds to an HLA class I or class II molecule and/or induces T-cells cross-reacting with said peptide, or a pharmaceutically acceptable salt thereof, wherein said peptide is not the underlying full-length polypeptide.

As used herein, the terms “identical” or percent “identity”, when used anywhere herein in the context of two or more nucleic acid or protein/polypeptide sequences, refer to two or more sequences or subsequences that are the same or have (or have at least) a specified percentage of amino acid residues or nucleotides that are the same (i.e., at, or at least, about 60% identity, preferably at, or at least, 65%, 70%, 75%, 80%, 85%, 90%, 91%, 92%, 93% or 94%, identity, and more preferably at, or at least, about 95%, 96%, 97%, 98%, 99%, or higher identity over a specified region—preferably over their full length sequences—, when compared and aligned for maximum correspondence over the comparison window or designated region) as measured using a sequence comparison algorithms, or by manual alignment and visual inspection (see, e.g., NCBI web site). In a particular embodiment, for example when comparing the protein or nucleic acid sequence of an antigen recognizing construct of the invention to another protein/gene, the percentage identity can be determined by the Blast searches using BLASTP 2.2.28+ with the following parameters: Matrix: BLOSUM62; Gap Penalties: Existence: 11, Extension: 1; Neighboring words threshold: 11; Window for multiple hits: 40.

In the context of the present invention it shall be understood that any embodiments referred to as “comprising” certain features of the invention, shall be understood to include in some more preferred embodiments the more restricted description of “consisting of” or “consisting essentially of” the very same features of the present invention.

In another additional or alternative embodiment, the antigen recognizing construct may further comprise a CDR1 and/or a CDR2 domain sequence. Within the variable domain, CDR1 and CDR2 are found in the variable (V) region of a polypeptide chain, and CDR3 includes some of V, all of diversity (D) and joining (J) regions. CDR3 is the most variable and is the main CDR responsible for specifically and selectively recognizing an antigen. CDR1 and CDR2 sequences may be selected from a CDR sequence of a human variable chain allele.

Native alpha-beta heterodimeric TCRs have an alpha chain and a beta chain. Each chain comprises variable, joining and constant regions, and the beta chain also usually contains a short diversity region between the variable and joining regions, but this diversity region is often considered as part of the joining region. Each variable region comprises three CDRs (Complementarity Determining Regions) embedded in a framework sequence, one being the hypervariable region named CDR3. There are several types of alpha chain variable (Vα) regions and several types of beta chain variable (Vβ) regions distinguished by their framework, CDR1 and CDR2 sequences, and by a partly defined CDR3 sequence. The Vα types are referred to in IMGT nomenclature by a unique TRAV number, Vβ types are referred to by a unique TRBV number. For more information on immunoglobulin antibody and TCR genes see the international ImMunoGeneTics information system®, Lefranc M-P et al (Nucleic Acids Res. 2015 January; 43(Database issue): D413-22; and http://www.imgt.org/).

For TCRs it is known that their capability of peptide binding is mainly mediated via the CDR3 region in the TCR variable chains. CDR1 and CDR2 regions are required for anchoring the TCR to the MHC chains presenting the peptide. Hence, for TCRs, it is preferred that a construct of the invention comprises a CDR3 as disclosed herein above, but wherein said CDR3 is provided in context of a functional TCR binding domain, which is characterized by the presence of at least CDR1 and CDR2 derived from a germline (human preferably), and any known functional TCR variable region framework region, preferably variable and constant human framework regions. Such sequences can be derived from the IMGT database as referenced herein above.

Therefore, in one additional or alternative embodiment the antigen recognizing construct of the invention comprises CDR1, CDR2 and CDR3 sequences in a combination as provided in table 1 herein below, or table 1a (table with the new sequences) herein below, which display the respective variable chain allele together with the CDR3 sequence. Therefore, preferred are antigen recognizing constructs of the invention which comprise at least one (preferably the CDR3), preferably, all three CDR sequences CDR1, CDR2 and CDR3. Preferably, an antigen recognizing construct of the invention comprises the respective CDR1 to CDR3 of one individual herein disclosed TCR variable region of the invention (see table 1 or table 1a herein below and the example section).

Preferably the TCR of the invention comprise an alpha chain or beta chain sequence, or variable region thereof, which are combined (one alpha one beta) according the indicated TCR number, for example combination of alpha and beta chains are possible among all respective chains indicated for TCR numbers 1376, 1377, 1378, 1375, 9283, 9386, 9651, 9652, and 3748, but preferably no combinations of chains are permitted of chains derived from different TCR numbers.

The term “specificity” or “antigen specificity” or “specific for” a given antigen, as used herein means that the antigen recognizing construct can specifically bind to said antigen, preferably a TSA antigen, more preferably with high avidity, when said antigen is presented by HLA, preferably by HLA-A*o2. For example, a TCR, as antigen recognizing construct, may be considered to have “antigenic specificity” for the TSA, if T cells expressing the TCR and contacted with a TSA presenting HLA secrete at least about 200 pg/ml or more (e.g., 250 pg/ml or more, 300 pg/ml or more, 400 pg/ml or more, 500 pg/ml or more, 600 pg/ml or more, 700 pg/ml or more, 1000 pg ml or more, 2,000 pg/ml or more, 2,500 pg/ml or more, 5,000 pg/ml or more) of interferon γ (IFN-γ) upon co-culture with target cells pulsed with a low concentration of a TSA antigen, such as the TSA epitopes and antigens provided herein below (e.g., about 10-11 mol/l, 10-10 mol/l, 10-9 mol/l, 10-8 mol/l, 10-7 mol/l, 10-6 mol/l, 10-5 mol/l). Alternatively, or additionally, a TCR may be considered to have “antigenic specificity” for the TSA, if T cells expressing the TCR secrete at least twice as much IFN-γ as the untransduced background level of IFN-γ upon co-culture with target cells pulsed with a low concentration of the TSA peptide antigens. Such a “specificity” as described above can—for example—be analyzed with an ELISA.

In one alternative or additional embodiment of the invention, the antigen recognizing construct selectively binds to a TSA derived antigenic peptide; preferably wherein the TSA antigenic peptide is a protein epitope or peptide having an amino acid sequence shown in SEQ ID NO: 76, 77, 78 or 163, or a variant thereof, wherein the variant is an amino acid deletion, addition, insertion or substitution of not more than three, preferably two and most preferably not more than one amino acid position. Preferably the antigenic peptide according to the invention is a spliced variant of the corresponding sequence of the parent antigen sequence. Preferably the TSA is the peptide according to SEQ ID NO: 76.

The term “selectivity” or “selective recognizing/binding” is understood to refer to the property of an antigen recognizing construct, such as a TCR or antibody, to selectively recognize or bind to preferably only one specific epitope and preferably shows no or substantially no cross-reactivity to another epitope. Preferably “selectivity” or “selective recognizing/binding” means that the antigen recognizing construct (e.g. a TCR) selectively recognizes or binds to preferably only one specific epitope and preferably shows no or substantially no cross-reactivity to another epitope, preferably no cross reactivity to the wild type not mutated ras oncogene product, wherein said epitope is unique for one protein, such that the antigen recognizing construct shows no or substantially no cross-reactivity to another epitope and another protein.

The antigen recognizing construct according to the invention is preferably selected from an antibody, or derivative or fragment thereof, or a T cell receptor (TCR), or derivative or fragment thereof. A derivative or fragment of an antibody or TCR of the invention shall preferably retain the antigen binding/recognizing ability of the parent molecule, in particular its specificity and/or selectivity as explained above. Such binding functionality may be retained by the presence of a CDR3 region as defined herein.

In an embodiment of the invention, the inventive TCRs are able to recognize TSA antigens in a major histocompatibility complex (MHC) class I-dependent manner. “MHC class I-dependent manner,” as used herein, means that the TCR elicits an immune response upon binding to TSA antigens within the context of an MHC class I molecule. The MHC class I molecule can be any MHC class I molecule known in the art, e.g., HLA-A molecules. In a preferred embodiment of the invention, the MHC class I molecule is an HLA-A*02 molecule, most preferably a A*02.01 molecule.

The invention provides both single chain antigen recognizing construct and double chain recognizing constructs.

In an embodiment, the TCR alpha variable domain has at least one mutation relative to a TCR alpha domain shown in Table 1 or table 1a; and/or the TCR beta variable domain has at least one mutation relative to a TCR alpha domain shown in Table 1 or table 1a. In an embodiment, a TCR comprising at least one mutation in the TCR alpha variable domain and/or TCR beta variable domain has a binding affinity for, and/or a binding half-life for, an TSA peptide-HLA molecule complex, which is at least double that of a TCR comprising the unmutated TCR alpha domain and/or unmutated TCR beta variable domain.

The TCR alpha chains of the present description may further comprise a TCR alpha transmembrane domain and/or a TCR alpha intracellular domain. The TCR beta chains of the present description may further comprise a TCR beta transmembrane domain and/or a TCR beta intracellular domain.

The invention in particular provides a TCR as antigen recognizing construct, or fragment or derivative thereof. The TCR preferably is of human, which is understood as being generated from a human TCR locus and therefore comprising human TCR sequences. Furthermore, the TCR of the invention may be characterized in that it is of human origin and specifically recognizes a TSA antigen of the invention.

Another embodiment of the invention additionally or alternatively provides the antigen recognizing construct described above, which induces an immune response, preferably wherein the immune response is characterized by an increase in interferon (IFN) γ levels.

TCRs of the invention may be provided as single chain α or β, or γ and δ, molecules, or alternatively as double chain constructs composed of both the α and β chain, or γ and δ chain.

The antigen recognizing construct of the invention may comprise a TCR α or γ chain; and/or a TCR β or δ chain; wherein the TCR α or γ chain comprises a CDR3 having at least 50%, 60%, 70%, 80%, 90%, 95%, 98%, 99%, or 100% sequence identity to an amino acid sequence selected from SEQ ID Nos. 3, 11, 19, 27, 35, 43, 51, 59, and 67, and/or wherein the TCR β or δ chain comprises a CDR3 having at least 50%, 60%, 70%, 80%, 90%, 95%, 98%, 99%, or 100% sequence identity to an amino acid sequence selected from SEQ ID Nos. 7, 15, 23, 31, 39, 47, 55, 63, and 71.

The antigen recognizing construct of the invention may also comprise a TCR α or γ chain; and/or a TCR β or δ chain; wherein the TCR α or γ chain comprises a CDR3 having at least 50%, 60%, 70%, 80%, 90%, 95%, 98%, 99%, or 100% sequence identity to an amino acid sequence selected from SEQ ID Nos. 93, 97, 101, 109, 113, 117, 121, 125, 141, 145, 149, and 153, and/or wherein the TCR β or δ chain comprises a CDR3 having at least 50%, 60%, 70%, 80%, 90%, 95%, 98%, 99%, or 100% sequence identity to an amino acid sequence selected from SEQ ID Nos. 85, 89, 105, 129, 133, 137, 157, and 161.

Most preferably, in some additional embodiments, wherein the disclosure refers to antigen recognizing constructs comprising any one, two or all of the CDR1 to CDR3 regions of the herein disclosed TCR chains (see table 1 or table 1a respectively), such antigen recognizing constructs may be preferred, which comprise the respective CDR sequence of the invention with not more than three, two, and preferably only one, modified amino acid residues. A modified amino acid residue may be selected from an amino acid insertion, deletion or substitution. Most preferred is that the three, two, preferably only one modified amino acid residue is the first or last amino acid residue of the respective CDR sequence. If the modification is a substitution then it is preferable in some embodiments that the substitution is a conservative amino acid substitution.

The TCR, or the antigen binding fragment thereof, is in some embodiments composed of a TCR α and a TCR β chain, or γ and δ chain. Such a double chain TCR comprises within each chain variable regions, and the variable regions each comprise one CDR1, one CDR2 and one CDR3 sequence. The TCRs comprises the CDR1 to CDR3 sequences as comprised in the variable chain amino acid sequence of as depicted in table 1 below.

Some embodiments of the invention pertain to a TCR, or a fragment thereof, composed of a TCR α and a TCR β chain, wherein said TCR comprises the variable region sequences having at least 50%, 60%, 70%, 80%, 90%, 95%, 98%, 99%, or preferably 100% sequence identity to the amino acid sequence selected from the α and β chain according to the following table A or A2:

A

| Alpha Chain Variable | Beta Chain Variable |
|---|---|
| Region (SEQ ID NO:) | Region (SEQ ID NO:) |
| 4 | 8 |
| 12 | 16 |
| 20 | 24 |
| 28 | 32 |
| 36 | 40 |
| 44 | 48 |
| 52 | 56 |
| 60 | 64 |
| 68 | 72 |

A2

| Alpha Chain Variable | Beta Chain Variable |
|---|---|
| Region (SEQ ID NO:) | Region (SEQ ID NO:) |
| 4 | 82 |
| 12 | 86 |
| 12 | 90 |
| 94 | 106 |
| 98 | 106 |
| 102 | 106 |
| 20 | 106 |
| 94 | 24 |
| 98 | 24 |
| 102 | 24 |
| 110 | 130 |
| 110 | 134 |
| 110 | 138 |
| 114 | 130 |
| 114 | 134 |
| 114 | 138 |
| 118 | 130 |
| 118 | 134 |
| 118 | 138 |
| 122 | 130 |
| 122 | 134 |
| 122 | 138 |
| 126 | 130 |
| 126 | 134 |
| 126 | 138 |
| 142 | 158 |
| 142 | 162 |
| 146 | 158 |
| 146 | 162 |
| 150 | 158 |
| 150 | 162 |
| 154 | 158 |
| 154 | 162 |

Mod 1376 (Seq ID79-82) is a possible variation in the CDR1! The inventive TCRs may further comprise a constant region derived from any suitable species, such as any mammal, e.g., human, rat, monkey, rabbit, donkey, or mouse. In an embodiment of the invention, the inventive TCRs further comprise a human constant region. In some preferred embodiments, the constant region of the TCR of the invention may be slightly modified, for example, by the introduction of heterologous sequences, preferably mouse sequences, which may increase TCR expression and stability.

Some embodiments of the invention pertain to a TCR, or a fragment thereof, composed of a TCR α and a TCR β chain, wherein said TCR comprises a human or mouse TCR constant region sequence. The constant domain may be human and partially murinized, for example in the extracellular part of the constant region. Other chimeric sequences may also be used for providing a TCR according to the present invention.

The TCR α or γ chain of the invention may further comprise a CDR1 having at least 50%, 60%, 70%, 80%, 90%, 95%, 98%, 99%, or 100% sequence identity to an amino acid sequence selected from SEQ ID Nos. 1, 9, 17, 25, 33, 41, 49, 57, and 65, or selected from an alpha chain CDR1 of table 1a; and/or a CDR2 having at least 50%, 60%, 70%, 80%, 90%, 95%, 98%, 99%, or 100% sequence identity to an amino acid sequence selected from SEQ ID Nos. 2, 10, 18, 26, 34, 42, 50, 58, and 66 or selected from an alpha chain CDR2 of table 1a.

According to the invention the TCR β or δ chain may further comprise a CDR1 having at least 50%, 60%, 70%, 80%, 90%, 95%, 98%, 99%, or 100% sequence identity to an amino acid sequence selected from SEQ ID Nos. 5, 13, 21, 29, 37, 45, 53, 61, and 69, or selected from a beta chain CDR1 of table 1a; and/or a CDR2 having at least 50%, 60%, 70%, 80%, 90%, 95%, 98%, 99%, or 100% sequence identity to an amino acid sequence selected from SEQ ID Nos. 6, 14, 22, 30, 38, 46, 54, 62, and 70, or selected from a beta chain CDR2 of table 1a.

The antigen recognizing construct may in a further embodiment comprise a binding fragment of a TCR, and wherein said binding fragment comprises CDR1 to CDR3, optionally selected from the CDR1 to CDR3 sequences having the amino acid sequences of SEQ ID Nos. 1 to 3, or 5 to 7, or 9 to 11, or 13 to 15, or 17 to 19, or 21 to 23, or 25 to 27, or 29 to 31, or 33 to 35, or 37 to 39, or 41 to 43, or 45 to 47, or 49 to 51, or 53 to 55, or 57 to 59, or 61 to 63, or 65 to 67, or 69 to 71, or selected from CDR1 to CDR2 of the any one of the chains of table 1a.

In further embodiments of the invention the antigen recognizing construct as described herein elsewhere is a TCR, or a fragment thereof, composed of at least one TCR α and one TCR β chain sequence, wherein said TCR α chain sequence and said TCR β chain sequence is selected from the following combinations of table B or B1:

B:

| Alpha Chain CDR1, CDR2, CDR3 | | | Beta Chain CDR1, CDR2, CDR3 | | |
|---|---|---|---|---|---|
| (SEQ ID NO) | | | (SEQ ID NO) | | |
| 1 | 2 | 3 | 5 | 6 | 7 |
| 9 | 10 | 11 | 13 | 14 | 15 |
| 17 | 18 | 19 | 21 | 22 | 23 |
| 25 | 26 | 27 | 29 | 30 | 31 |
| 33 | 34 | 35 | 37 | 38 | 39 |
| 41 | 42 | 43 | 45 | 46 | 47 |
| 49 | 50 | 51 | 53 | 54 | 55 |
| 57 | 58 | 59 | 61 | 62 | 63 |
| 65 | 66 | 67 | 69 | 70 | 71 |

B1

| Alpha Chain CDR1, CDR2, CDR3 | | | Beta Chain CDR1, CDR2, CDR3 | | |
|---|---|---|---|---|---|
| (SEQ ID NO) | | | (SEQ ID NO) | | |
| 1 | 2 | 3 | 79 | 80 | 81 |
| 9 | 10 | 11 | 83 | 84 | 85 |
| 9 | 10 | 11 | 87 | 88 | 89 |
| 91 | 92 | 93 | 103 | 104 | 105 |
| 95 | 96 | 97 | 103 | 104 | 105 |
| 99 | 100 | 101 | 103 | 104 | 105 |
| 17 | 18 | 19 | 103 | 104 | 105 |
| 91 | 92 | 93 | 21 | 22 | 23 |
| 95 | 96 | 97 | 21 | 22 | 23 |
| 99 | 100 | 101 | 21 | 22 | 23 |
| 107 | 108 | 109 | 127 | 128 | 129 |
| 107 | 108 | 109 | 131 | 132 | 133 |
| 107 | 108 | 109 | 135 | 136 | 137 |
| 111 | 112 | 113 | 127 | 128 | 129 |
| 111 | 112 | 113 | 131 | 132 | 133 |
| 111 | 112 | 113 | 135 | 136 | 137 |
| 115 | 116 | 117 | 127 | 128 | 129 |
| 115 | 116 | 117 | 131 | 132 | 133 |
| 115 | 116 | 117 | 135 | 136 | 137 |
| 119 | 120 | 121 | 127 | 128 | 129 |
| 119 | 120 | 121 | 131 | 132 | 133 |
| 119 | 120 | 121 | 135 | 136 | 137 |
| 123 | 124 | 125 | 127 | 128 | 129 |
| 123 | 124 | 125 | 131 | 132 | 133 |
| 123 | 124 | 125 | 135 | 136 | 137 |
| 139 | 140 | 141 | 155 | 156 | 157 |
| 139 | 140 | 141 | 159 | 160 | 161 |
| 143 | 144 | 145 | 155 | 156 | 157 |
| 143 | 144 | 145 | 159 | 160 | 161 |
| 147 | 148 | 149 | 155 | 156 | 157 |
| 147 | 148 | 149 | 159 | 160 | 161 |
| 151 | 152 | 153 | 155 | 156 | 157 |
| 151 | 152 | 153 | 159 | 160 | 161 |

In further embodiments of the invention the antigen recognizing construct as described herein before is a TCR, or a fragment thereof, comprising at least one TCRα and one TCR β chain sequence, wherein said TCR α chain sequence and said TCR β chain sequence is selected from the combinations of alpha and beta chains sequences in table A.

As used herein, the term “murine” or “human,” when referring to an antigen recognizing construct, or a TCR, or any component of a TCR described herein (e.g., complementarity determining region (CDR), variable region, constant region, α chain, and/or β chain), means a TCR (or component thereof), which is derived from a unrearranged human TCR locus of a mouse or a human, respectively.

In an embodiment of the invention, chimeric TCR are provided, wherein the TCR chains comprise sequences from multiple species. Preferably, a TCR of the invention may comprise an a chain comprising a human variable region of an a chain and, for example, a murine constant region of a murine TCR α chain.

In one embodiment, the TCR of the invention is a human TCR comprising human variable regions according to the above embodiments and human constant regions.

In some embodiments the antigen recognizing construct is murinized or humanized. These terms are used when amino acid sequences from a foreign species are introduced into a construct of the invention.

The TCR of the invention may be provided as a single chain TCR (scTCR). A scTCR can comprise a polypeptide of a variable region of a first TCR chain (e.g., an alpha chain) and a polypeptide of an entire (full-length) second TCR chain (e.g., a beta chain), or vice versa. Furthermore, the scTCR can optionally comprise one or more linkers which join the two or more polypeptides together. The linker can be, for instance, a peptide, which joins together two single chains, as described herein. Also provided is such a scTCR of the invention, which is fused to a human cytokine, such as IL-2, IL-7 or IL-15.

The antigen recognizing construct according to the invention can also be provided in the form of a multimeric complex, comprising at least two scTCR molecules, wherein said scTCR molecules are each fused to at least one biotin moiety, or other interconnecting molecule/linker, and wherein said scTCRs are interconnected by biotin-streptavidin interaction to allow the formation of said multimeric complex. Similar approaches known in the art for the generation of multimeric TCR are also possible and included in this disclosure. Also provided are multimeric complexes of a higher order, comprising more than two scTCR of the invention.

For the purposes of the present invention, a TCR is a moiety having at least one TCR alpha or gamma and/or TCR beta or delta variable domain. Generally, they comprise both a TCR alpha variable domain and a TCR beta variable domain, alternatively both a TCR gamma variable domain and a TCR delta variable domain. They may be αβ/γδ heterodimers or may be in single chain format. For use in adoptive therapy, an αβ or γδ heterodimeric TCR may, for example, be transfected as full-length chains having both cytoplasmic and transmembrane domains. If desired, an introduced disulfide bond between residues of the respective constant domains may be present.

In a preferred embodiment, the antigen recognizing construct is a human TCR, or fragment or derivative thereof. A human TCR or fragment or derivative thereof is a TCR, which comprises over 50% of the corresponding human TCR sequence. Preferably, only a small part of the TCR sequence is of artificial origin or derived from other species. It is known, however, that chimeric TCRs, e.g. derived from human origin with murine sequences in the constant domains, are advantageous. Particularly preferred are, therefore, TCRs in accordance with the present invention, which contains murine sequences in the extracellular part of their constant domains.

Thus, it is also preferred that the inventive antigen recognizing construct is able to recognize its antigen in a human leucocyte antigen (HLA) dependent manner, preferably in a HLA-A02 dependent manner. The term “HLA dependent manner” in the context of the present invention means that the antigen recognizing construct binds to the antigen only in the event that the antigenic peptide is presented by said HLA.

The antigen recognizing construct in accordance with the invention in one embodiment preferably induces an immune response, preferably wherein the immune response is characterized by the increase in interferon (IFN) γ levels.

Also provided by the invention is a polypeptide comprising a functional portion of any of the TCRs (or functional variants thereof) described herein, for examples, of any one of the TCRs selected from the TCR provided in the example section and table 1. The term “polypeptide” as used herein includes oligopeptides and refers to a single chain of amino acids connected by one or more peptide bonds. With respect to the inventive polypeptides, the functional portion can be any portion comprising contiguous amino acids of the TCR (or functional variant thereof), of which it is a part, provided that the functional portion specifically binds to the TSA antigen, preferably as disclosed herein in table 2 (SEQ ID NOs: 76 to 78, and 163). The term “functional portion” when used in reference to a TCR (or functional variant thereof) refers to any part or fragment of the TCR (or functional variant thereof) of the invention, which part or fragment retains the biological activity of the TCR (or functional variant thereof), of which it is a part (the parent TCR or parent functional variant thereof). Functional portions encompass, for example, those parts of a TCR (or functional variant thereof) that retain the ability to specifically bind to the TSA antigen (in an HLA dependent manner), or detect, treat, or prevent cancer, to a similar extent, the same extent, or to a higher extent, as the parent TCR (or functional variant thereof). In reference to the parent TCR (or functional variant thereof), the functional portion can comprise, for instance, about 10%, 25%, 30%, 50%, 68%, 80%, 90%, 95%, or more, of the parent TCR variable sequences (or functional variant thereof).

The functional portion can comprise additional amino acids at the amino or carboxy terminus of the portion, or at both termini, in which additional amino acids are not found in the amino acid sequence of the parent TCR or functional variant thereof. Desirably, the additional amino acids do not interfere with the biological function of the functional portion, e.g., specifically binding to the TAA antigens; and/or having the ability to detect cancer, treat or prevent cancer, etc. More desirably, the additional amino acids enhance the biological activity, as compared to the biological activity of the parent TCR or functional variant thereof.

The polypeptide can comprise a functional portion of either or both of the α and β chains of the TCRs or functional variant thereof of the invention, such as a functional portion comprising one of more of CDR1, CDR2, and (preferably) CDR3 of the variable region(s) of the α chain and/or β chain of a TCR or functional variant thereof of the invention. In an embodiment of the invention, the polypeptide can comprise a functional portion comprising the amino acid sequence of SEQ ID NO: 3, 7, 11, 15, 19, 23, 27, 31, 35, 39, 43, 47, 51, 55, 59, 63, 67, and 71 (CDR3 of the variable regions of the TCR of the invention), or a combination thereof. In an embodiment of the invention, the inventive polypeptide can comprise, for instance, the variable region of the inventive TCR or functional variant thereof comprising a combination of the CDR regions set forth above. In this regard, the polypeptide can comprise the amino acid sequence of any of SEQ ID NO: 4, 8, 12, 16, 20, 24, 28, 32, 36, 40, 44, 48, 52, 56, 60, 64, 68, and 72 (the variable regions of an α or β chain of the TCR of the invention).

The polypeptide can also comprise a functional portion of either or both of the α and β chains of the TCRs or functional variant thereof of the invention, such as a functional portion comprising one of more of CDR1, CDR2, and (preferably) CDR3 of the variable region(s) of the α chain and/or β chain of a TCR or functional variant thereof of the invention. In an embodiment of the invention, the polypeptide can comprise a functional portion comprising the amino acid sequence of SEQ ID NO: 85, 89, 93, 97, 101, 105, 109, 113, 117, 121, 125, 129, 133, 137, 141, 145, 149, 153, 157, and 161 (CDR3 of the variable regions of the TCR of the invention according to table 1a), or a combination thereof. In an embodiment of the invention, the inventive polypeptide can comprise, for instance, the variable region of the inventive TCR or functional variant thereof comprising a combination of the CDR regions set forth above. In this regard, the polypeptide can comprise the amino acid sequence of any of SEQ ID NO: 86, 90, 94, 98, 102, 106, 110, 114, 118, 122, 126, 130, 134, 138, 142, 146, 150, 154, 158, and 162 (the variable regions of an α or β chain of the TCR of the invention according to table 1a below).

The inventive TCR may in addition comprise constant domain sequences (preferably human) or functional variants of constant domain sequences.

In some instances, the construct of the invention may comprise one or two polypeptide chains comprising a sequences according to any of the SEQ ID NO: 1 to 72, and 79 to162 (CDR sequences, and variable regions), or functional fragments thereof, and further comprise(s) other amino acid sequences, e.g., an amino acid sequence encoding an immunoglobulin or a portion thereof, then the inventive protein can be a fusion protein. In this regard, the invention also provides a fusion protein comprising at least one of the inventive polypeptides described herein along with at least one other polypeptide. The other polypeptide can exist as a separate polypeptide of the fusion protein, or can exist as a polypeptide, which is expressed in frame (in tandem) with one of the inventive polypeptides described herein. The other polypeptide may include any peptidic or proteinaceous molecule, or a portion thereof, including, but not limited to an immunoglobulin, CD3, CD4, CD8, an MHC molecule, a CD1 molecule, e.g., CD1a, CD1b, CD1c, CD1d, etc.

The fusion protein can comprise one or more copies of the inventive polypeptide and/or one or more copies of the other polypeptide. For instance, the fusion protein can comprise 1, 2, 3, 4, 5, or more, copies of the inventive polypeptide and/or of the other polypeptide. Suitable methods of making fusion proteins are known in the art, and include, for example, recombinant methods. In some embodiments of the invention, the TCRs (and functional portions and functional variants thereof), polypeptides, and proteins of the invention may be expressed as a single protein comprising a linker peptide linking the α chain and the β chain, and linking the γ chain and the δ chain. In this regard, the TCRs (and functional variants and functional portions thereof), polypeptides, and proteins of the invention comprising the amino acid sequences of the variable regions of the TCR of the invention and may further comprise a linker peptide. The linker peptide may advantageously facilitate the expression of a recombinant TCR (including functional portions and functional variants thereof), polypeptide, and/or protein in a host cell. The linker peptide may comprise any suitable amino acid sequence. Linker sequences for single chain TCR constructs are well known in the art. Such a single chain construct may further comprise one, or two, constant domain sequences. Upon expression of the construct including the linker peptide by a host cell, the linker peptide may also be cleaved, resulting in separated α and β chains, and separated γ and δ chain.

As already mentioned above, the binding functionality of the TCR of the invention may be provided in the framework of an antibody. For example, CDR sequences of the TCR of the invention, possibly including additional 3, 2 or 1 N and/or c terminal framework residues, may be directly grafted into an antibody variable heavy/light chain sequence. The term “antibody” in its various grammatical forms is used herein to refer to immunoglobulin molecules and immunologically active portions of immunoglobulin molecules, i.e., molecules that contain an antigen-binding site or a paratope. Such molecules are also referred to as “antigen binding fragments” of immunoglobulin molecules. The invention further provides an antibody, or antigen binding portion thereof, which specifically binds to the antigens described herein. The antibody can be any type of immunoglobulin that is known in the art. For instance, the antibody can be of any isotype, e.g., IgA, IgD, IgE, IgG, IgM, etc. The antibody can be monoclonal or polyclonal. The antibody can be a naturally-occurring antibody, e.g., an antibody isolated and/or purified from a mammal, e.g., mouse, rabbit, goat, horse, chicken, hamster, human, etc. Alternatively, the antibody can be a genetically-engineered antibody, e.g., a humanized antibody or a chimeric antibody. The antibody can be in monomeric or polymeric form.

The term “antibody” includes, but is not limited to, genetically engineered or otherwise modified forms of immunoglobulins, such as intrabodies, chimeric antibodies, fully human antibodies, humanized antibodies (e.g. generated by “CDR-grafting”), antibody fragments, and heteroconjugate antibodies (e.g., bispecific antibodies, diabodies, triabodies, tetra-bodies, etc.). The term “antibody” includes cys-diabodies and minibodies. Thus, each and every embodiment provided herein in regard to “antibodies”, or “antibody like constructs” is also envisioned as, bi-specific antibodies, diabodies, scFv fragments, chimeric antibody receptor (CAR) constructs, diabody and/or minibody embodiments, unless explicitly denoted otherwise. The term “antibody” includes a polypeptide of the immunoglobulin family or a polypeptide comprising fragments of an immunoglobulin that is capable of non-covalently, reversibly, and in a specific manner binding a corresponding antigen, preferably the TSA of the invention, as disclosed herein. An exemplary antibody structural unit comprises a tetramer. In some embodiments, a full length antibody can be composed of two identical pairs of polypeptide chains, each pair having one “light” and one “heavy” chain (connected through a disulfide bond). Antibody structure and isotypes are well known to the skilled artisan (for example from Janeway's Immunobiology, 9th edition, 2016).

The recognized immunoglobulin genes of mammals include the kappa, lambda, alpha, gamma, delta, epsilon, and mu constant region genes, as well as the myriad immunoglobulin variable region genes (for more information on immunoglobulin genes see the international ImMunoGeneTics information system®, Lefranc M-P et al, Nucleic Acids Res. 2015 January; 43 (Database issue): D413-22; and http://www.imgt.org/). For full-length chains, the light chains are classified as either kappa or lambda. For full-length chains, the heavy chains are classified as gamma, mu, alpha, delta, or epsilon, which in turn define the immunoglobulin classes, IgG, IgM, IgA, IgD, and IgE, respectively. The N-terminus of each chain defines a variable region of about 100 to 110 or more amino acids primarily responsible for antigen recognition. The terms variable light chain (VL) and variable heavy chain (VH) refer to these regions of light and heavy chains respectively. As used in this invention, an “antibody” encompasses all variations of antibody and fragments thereof. Thus, within the scope of this concept are full length antibodies, chimeric antibodies, humanized antibodies, single chain antibodies (scFv), Fab, Fab′, and multimeric versions of these fragments (e.g., F(ab′)2) with the same, essentially the same or similar binding specificity. In some embodiments, the antibody binds specifically to a peptide TAA of the invention. Preferred antigen recognizing constructs according to the invention include an antibody heavy chain, preferably the variable domain thereof, or an antigen binding fragment thereof, and/or an antibody light chain, preferably the variable domain thereof, or an antigen binding fragment thereof. Similarly, disulfide-stabilized variable region fragments (dsFv) can be prepared by recombinant DNA technology, antibody fragments of the invention, however, are not limited to these exemplary types of antibody fragments. Also, the antibody, or antigen binding portion thereof, can be modified to comprise a detectable label, such as, for instance, a radioisotope, a fluorophore (e.g., fluorescein isothiocyanate (FITC), phycoerythrin (PE)), an enzyme (e.g., alkaline phosphatase, horseradish peroxidase), and element particles (e.g., gold particles). In some instances, the TCR CDR3 sequence may be slightly modified, but preferably by not more than 3 amino acid residues, preferably only two and most preferably only one amino acid position, as compared to the CDR3 sequences provided in SEQ ID Nos: 3, 7, 11, 15, 19, 23, 27, 31, 35, 39, 43, 47, 51, 55, 59, 63, 67, and 71, or any other CDR3 shown in table 1a. Preferably, the antibodies comprise the CDR3, preferably all of CDR1 to CDR3 regions in the combination, as indicated for the TCR of the invention in table 1 or table 1a, in each case independently, optionally with not more than three or two, preferably one, amino acid substitution(s), insertion(s) and/or deletion(s) compared to these sequences.

Suitable methods of making antibodies are known in the art. For instance, standard hybridoma methods are described in, e.g., Kohler and Milstein, Eur. J. Immunol, 5, 511-519 (1976), Harlow and Lane (eds.), Antibodies: A Laboratory Manual, CSH Press (1988), and C. A. Janeway et al. (eds.), Immunobiology, 8 Ed., Garland Publishing, New York, N.Y. (201 1)). Alternatively, other methods, such as EBV-hybridoma methods (Haskard and Archer, J. Immunol. Methods, 74(2), 361-67 (1984), and Roder et al, Methods Enzymol, 121, 140-67 (1986)), and bacteriophage vector expression systems (see, e.g., Huse et al., Science, 246, 1275-81 (1989)) are known in the art. Further, methods of producing antibodies in non-human animals are described in, e.g., U.S. Pat. Nos. 5,545,806, 5,569,825, and 5,714,352, and U.S. Patent Application Publication No. 2002/0197266.

Some embodiments of the invention also pertain to TCRs, or functional fragments and polypeptides thereof, which are soluble TCRs. As used herein, the term “soluble T-cell receptor” refers to heterodimeric truncated variants of native TCRs, which comprise extracellular portions of the TCR α-chain and β-chain, for example linked by a disulfide bond, but which lack the transmembrane and cytosolic domains of the native protein. The terms “soluble T-cell receptor α-chain sequence and soluble T-cell receptor β-chain sequence” refer to TCR α-chain and β-chain sequences that lack the transmembrane and cytosolic domains. The sequence (amino acid or nucleic acid) of the soluble TCR α-chain and β-chains may be identical to the corresponding sequences in a native TCR or may comprise variant soluble TCR α-chain and β-chain sequences, as compared to the corresponding native TCR sequences. The term “soluble T-cell receptor” as used herein encompasses soluble TCRs with variant or non-variant soluble TCR α-chain and β-chain sequences. The variations may be in the variable or constant regions of the soluble TCR α-chain and β-chain sequences and can include, but are not limited to, amino acid deletion, insertion, substitution mutations as well as changes to the nucleic acid sequence, which do not alter the amino acid sequence. Soluble TCR of the invention in any case retain the binding functionality of their parent molecules.

The above problem is further solved by a nucleic acid encoding for an antigen recognizing construct of the invention, or any of the aforementioned protein or polypeptide constructs. The nucleic acid preferably (a) has a strand encoding for an antigen recognizing construct according to the invention; (b) has a strand complementary to the strand in (a); or (c) has a strand that hybridizes under stringent conditions with a molecule as described in (a) or (b). Stringent conditions are known to the person of skill in the art, specifically from Sambrook et al, “Molecular Cloning”. In addition to that, the nucleic acid optionally has further sequences, which are necessary for expressing the nucleic acid sequence corresponding to the protein, specifically for expression in a mammalian/human cell. The nucleic acid used can be contained in a vector suitable for allowing expression of the nucleic acid sequence corresponding to the peptide in a cell. However, the nucleic acids can also be used to transform an antigen-presenting cell, which may not be restricted to classical antigen-presenting cells, such as dendritic cells, in such a way that they themselves produce the corresponding proteins on their cellular surface.

In some embodiments, the polypeptides of the antigen recognizing constructs can be encoded by nucleic acids and expressed in vivo or in vitro. Thus, in some embodiments, a nucleic acid encoding an antigen recognizing construct is provided. In some embodiments, the nucleic acid encodes one part or monomer of an antigen recognizing construct of the invention (for example one of two chains of a TCR of the invention), and/or another nucleic acid encodes another part or monomer of an antigen recognizing construct of the invention (for example the other of two chains of the TCR). In some embodiments, the nucleic acid encodes two or more antigen recognizing construct polypeptide chains, for example, at least 2 TCR chains. Nucleic acids encoding multiple antigen recognizing construct chains can include nucleic acid cleavage sites between at least two chain sequences, can encode transcription or translation start site between two or more chains sequences, and/or can encode proteolytic target sites between two or more antigen recognizing construct chains.

By “nucleic acid” as used herein includes “polynucleotide,” “oligonucleotide,” and “nucleic acid molecule,” and generally means a polymer of DNA or RNA, which can be single-stranded or double-stranded, synthesized or obtained (e.g., isolated and/or purified) from natural sources, which can contain natural, non-natural or altered nucleotides, and can contain a natural, non-natural or altered internucleotide linkage, such as a phosphoroamidate linkage or a phosphorothioate linkage, instead of the phosphodiester found between the nucleotides of an unmodified oligonucleotide.

Preferably, the nucleic acids of the invention are recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication. The nucleic acid can comprise any nucleotide sequence, which encodes any of the TCRs, polypeptides, or proteins, or functional portions or functional variants thereof described herein.

Furthermore, the invention provides a vector comprising a nucleic acid in accordance to the invention as described above. Desirably, the vector is an expression vector or a recombinant expression vector. The term “recombinant expression vector” refers in context of the present invention to a nucleic acid construct that allows for the expression of an mRNA, protein or polypeptide in a suitable host cell. The recombinant expression vector of the invention can be any suitable recombinant expression vector, and can be used to transform or transfect any suitable host. Suitable vectors include those designed for propagation and expansion or for expression or both, such as plasmids and viruses. Examples of animal expression vectors include pMP71, pEUK-Cl, pMAM, and pMAMneo. Preferably, the recombinant expression vector is a viral vector, e.g., a retroviral vector. The recombinant expression vector comprises regulatory sequences, such as transcription and translation initiation and termination codons, which are specific to the type of host cell (e.g., bacterium, fungus, plant, or animal), into which the vector is to be introduced and in which the expression of the nucleic acid of the invention may be performed. Furthermore, the vector of the invention may include one or more marker genes, which allow for selection of transformed or transfected hosts. The recombinant expression vector can comprise a native or normative promoter operably linked to the nucleotide sequence encoding the constructs of the invention, or to the nucleotide sequence, which is complementary to or which hybridizes to the nucleotide sequence encoding the constructs of the invention. The selections of promoters include, e.g., strong, weak, inducible, tissue-specific and developmental-specific promoters. The promoter can be a non-viral promoter or a viral promoter. The inventive recombinant expression vectors can be designed for either transient expression, for stable expression, or for both. Also, the recombinant expression vectors can be made for constitutive expression or for inducible expression.

The invention also pertains to a host cell comprising an antigen recognizing construct in accordance with the invention. Specifically, the host cell of the invention comprises a nucleic acid, or a vector as described herein above. The host cell can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. For purposes of producing a recombinant TCR, polypeptide, or protein, the host cell is preferably a mammalian cell. Most preferably, the host cell is a human cell. However, for protein production the host cell is preferably a Chinese hamster ovary (CHO) cell. While the host cell can be of any cell type, can originate from any type of tissue, and can be of any developmental stage, the host cell preferably is a peripheral blood leukocyte (PBL) or a peripheral blood mononuclear cell (PBMC). More preferably, the host cell is a T cell. The T cell can be any T cell, such as a cultured T cell, e.g., a primary T cell, or a T cell from a cultured T cell line, e.g., Jurkat, SupT1, etc., or a T cell obtained from a mammal, preferably a T cell or T cell precursor from a human patient. If obtained from a mammal, the T cell can be obtained from numerous sources, including but not limited to blood, bone marrow, lymph node, the thymus, or other tissues or fluids. T cells can also be enriched for or purified. Preferably, the T cell is a human T cell. More preferably, the T cell is a T cell isolated from a human. The T cell can be any type of T cell and can be of any developmental stage, including but not limited to, CD4-positive and/or CD8-positive, CD4-positive helper T cells, e.g., Th1 and Th2 cells, CD8-positive T cells (e.g., cytotoxic T cells), tumor infiltrating cells (TILs), memory T cells, naive T cells, and the like. Preferably, the T cell is a CD8-positive T cell or a CD4-positive T cell.

Preferably, the host cell of the invention is a lymphocyte, preferably, a T lymphocyte, such as a CD4-positive or CD8-positive T-cell. The host cell furthermore preferably is a tumor reactive T cell specific for TSA expressing tumor cells.

The objective of the invention is also solved by a method of manufacturing a TSA specific antigen recognizing construct, or of a TAA/TSA specific antigen recognizing construct expressing cell line, comprising

a. Providing a suitable host cell,

b. Providing a genetic construct comprising a coding sequence encoding for an antigen recognizing construct according to the herein disclosed invention,

c. Introducing into said suitable host cell said genetic construct, and

d. Expressing said genetic construct by said suitable host cell.

The method may further comprise a step of cell surface presentation of said antigen recognizing construct on said suitable host cell.

In other preferred embodiments, the genetic construct is an expression construct comprising a promoter sequence operably linked to said coding sequence.

Preferably, said antigen recognizing construct is of mammalian origin, preferably of human origin. The preferred suitable host cell for use in the method of the invention is a mammalian cell, such as a human cell, in particular a human T lymphocyte. T cells for use in the invention are described in detail herein above.

Also encompassed by the invention are embodiments, wherein said antigen recognizing construct is a modified TCR, wherein said modification is the addition of functional domains, such as a label or a therapeutically active substance. Furthermore, encompassed are TCR having alternative domains, such as an alternative membrane anchor domain instead of the endogenous transmembrane region.

Desirably, the transfection system for introducing the genetic construct into said suitable host cell is a retroviral or lentiviral vector system. Such systems are well known to the skilled artisan.

Also comprised by the present invention is in one embodiment the additional method step of isolation and purification of the antigen recognizing construct from the cell and, optionally, the reconstitution of the translated antigen recognizing construct-fragments in a T-cell.

In an alternative aspect of the invention a T-cell is provided obtained or obtainable by a method for the production of a T cell receptor (TCR), which is specific for tumorous cells and has high avidity as described herein above. Such a T cell is depending on the host cell used in the method of the invention, for example, a human or non-human T-cell, preferably a human TCR.

The term “isolated” as used herein in the context of a polypeptide, such as an antigen recognizing construct (an example of which could be an antibody), refers to a polypeptide that is purified from proteins or polypeptides or other contaminants that would interfere with its therapeutic, diagnostic, prophylactic, research or other use. An antigen recognizing construct according to the invention may be a recombinant, synthetic or modified (non-natural) antigen binding construct. The term “isolated” as used herein in the context of a nucleic acid or cells refers to a nucleic acid or cells that is/are purified from DNA, RNA, proteins or polypeptides or other contaminants (such as other cells) that would interfere with its therapeutic, diagnostic, prophylactic, research or other use, or it refers to a recombinant, synthetic or modified (non-natural) nucleic acid. In this context, a “recombinant” protein/polypeptide or nucleic acid is one made using recombinant techniques. Methods and techniques for the production of recombinant nucleic acids and proteins are well known in the art.

Treatment Methods and Diseases

One further aspect of the present invention relates to the herein disclosed antigen recognizing constructs, nucleic acids, vectors, pharmaceutical compositions and/or host cell for use in medicine. The use in medicine in one preferred embodiment includes the use in the diagnosis, prevention and/or treatment of a tumor disease, such as a malignant or benign tumor disease. The tumor disease is, for example, a tumor disease characterized by the expression of the TSA, in a cancer or tumor cell of said tumor disease. Preferably the tumor treatable according to the invention is a cancer positive for a mutated Ras, most preferably the Ras^{G12V} antigen and its spliced variant.

With respect to the above mentioned medical applications of the antigen recognizing constructs and other materials derived therefrom, pertaining thereto or encoding the same, in accordance of the present disclosure, the to be treated and/or to be diagnosed diseases can be any proliferative disorder or infectious disease, preferably characterized by the expression of the TAA/TSA or TAA/TSA epitope sequence of the invention, for example any cancer, including any of acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vagina, cancer of the vulva, chronic lymphocytic leukemia, chronic myeloid cancer, colon cancer, esophageal cancer, cervical cancer, gastrointestinal carcinoid tumor, glioma, Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, cancer of the oropharynx, ovarian cancer, cancer of the penis, pancreatic cancer, peritoneum, omentum, and mesentery cancer, pharynx cancer, prostate cancer, rectal cancer, renal cancer, skin cancer, small intestine cancer, soft tissue cancer, stomach cancer, testicular cancer, thyroid cancer, cancer of the uterus, ureter cancer, and urinary bladder cancer. A preferred cancer is a Ras^{G12V} positive cancer, more preferably a cancer positive for a peptide spliced variant of Ras^{G12V}, most preferably the cancer in at least one tumor cell, comprises and/or presents a peptide epitope according to any one of SEQ ID NO: 73 to 78, and 163, preferably 76 to 78, and 163, most preferably 76.

The constructs, proteins, TCRs antibodies, polypeptides and nucleic acids of the invention are in particular for use in immune therapy, preferably, in adoptive T cell therapy. The administration of the compounds of the invention can, for example, involve the infusion of T cells of the invention into said patient. Preferably, such T cells are autologous T cells of the patient and in vitro transduced with a nucleic acid or antigen recognizing construct of the present invention.

The inventive antigen recognizing constructs, TCRs, polypeptides, proteins (including functional variants thereof), nucleic acids, recombinant expression vectors, host cells (including populations thereof), and antibodies (including antigen binding portions thereof), all of which are collectively referred to as “inventive TCR materials” hereinafter, can be formulated into a composition, such as a pharmaceutical composition. In this regard, the invention provides a pharmaceutical composition comprising any of the antigen recognizing constructs, TCRs, polypeptides, proteins, functional portions, functional variants, nucleic acids, expression vectors, host cells (including populations thereof), and antibodies (including antigen binding portions thereof) described herein, and a pharmaceutically acceptable carrier, excipient and/or stabilizer. The inventive pharmaceutical compositions containing any of the inventive TCR materials can comprise more than one inventive TCR material, e.g., a polypeptide and a nucleic acid, or two or more different TCRs (including functional portions and functional variants thereof). Alternatively, the pharmaceutical composition can comprise an inventive TCR material in combination with another pharmaceutically active agent(s) or drug(s), such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc. Preferably, the carrier is a pharmaceutically acceptable carrier. With respect to pharmaceutical compositions, the carrier can be any of those conventionally used for the particular inventive TCR material under consideration. Such pharmaceutically acceptable carriers are well-known to those skilled in the art and are readily available to the public. It is preferred that the pharmaceutically acceptable carrier be one, which has no detrimental side effects or toxicity under the conditions of use.

Thus also provided is a pharmaceutical composition, comprising any of the herein described products of the invention and TCR materials of the invention, specifically any proteins, nucleic acids or host cells. In a preferred embodiment the pharmaceutical composition is for immune therapy, preferably adoptive cell therapy.

Preferably, the inventive TCR material is administered by injection, e.g., intravenously. When the inventive TCR material is a host cell expressing the inventive TCR (or functional variant thereof), the pharmaceutically acceptable carrier for the cells for injection may include any isotonic carrier such as, for example, normal saline (about 0.90% w/v of NaCl in water, about 300 mOsm/L NaCl in water, or about 9.0 g NaCl per liter of water), NORMOSOL R electrolyte solution (Abbott, Chicago, Ill.), PLASMA-LYTE A (Baxter, Deerfield, Ill.), about 5% dextrose in water, or Ringer's lactate. In an embodiment, the pharmaceutically acceptable carrier is supplemented with human serum albumen.

For purposes of the invention, the amount or dose (e.g., numbers of cells when the inventive TCR material is one or more cells) of the inventive TCR material administered may be sufficient to affect, e.g., a therapeutic or prophylactic response, in the subject or animal over a reasonable time frame. For example, the dose of the inventive TCR material should be sufficient to bind to a cancer antigen, or detect, treat or prevent cancer in a period of from about 2 hours or longer, e.g., 12 to 24 or more hours, from the time of administration. In certain embodiments, the time period could be even longer. The dose will be determined by the efficacy of the particular inventive TCR material and the condition of the animal (e.g., human), as well as the body weight of the animal (e.g., human) to be treated.

It is contemplated that the inventive pharmaceutical compositions, antigen recognizing constructs, TCRs (including functional variants thereof), polypeptides, proteins, nucleic acids, recombinant expression vectors, host cells, or populations of cells can be used in methods of treating or preventing cancer, or TSA-positive premalignancy. The inventive TCRs (and functional variants thereof) are believed to bind specifically to the TSA of the invention, such that the TCR (or related inventive polypeptide or protein and functional variants thereof), when expressed by or on a cell, such as a T cell, is able to mediate an immune response against a target cell expressing the TSA of the invention, preferably presenting TSA peptides via MHC I or II on the surface of said target cell. In this regard, the invention provides a method of treating or preventing a condition, in particular cancer, in a mammal, comprising administering to the mammal any of the pharmaceutical compositions, antigen recognizing constructs, in particular TCRs (and functional variants thereof), polypeptides, or proteins described herein, any nucleic acid or recombinant expression vector comprising a nucleotide sequence encoding any of the TCRs (and functional variants thereof), polypeptides, proteins described herein, or any host cell or population of cells comprising a nucleic acid or recombinant vector, which encodes any of the constructs of the invention (and functional variants thereof), polypeptides, or proteins described herein, in an amount effective to treat or prevent the condition in the mammal, wherein the condition is preferably cancer, such as a cancer expressing the TSA of the invention.

Examples of pharmaceutically acceptable carriers or diluents useful in the present invention include stabilizers such as SPGA, carbohydrates (e.g. sorbitol, mannitol, starch, sucrose, glucose, dextran), proteins such as albumin or casein, protein containing agents such as bovine serum or skimmed milk and buffers (e.g. phosphate buffer).

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of a condition in a mammal. Furthermore, the treatment or prevention provided by the inventive method can include treatment or prevention of one or more conditions or symptoms of the condition, e.g., cancer, being treated or prevented. For example, treatment or prevention can include promoting the regression of a tumor. Also, for purposes herein, “prevention” can encompass delaying the onset of the condition, or a symptom or condition thereof.

The present invention also relates to a method of treating cancer comprising administering a TCR, a nucleic acid, or a host cell of the present description in combination with at least one chemotherapeutic agent and/or radiation therapy.

Another aspect of the invention further pertains to a method for detecting a TSA protein, or a complex of MHC and the TSA protein (protein epitope of the TSA), in a (biological) sample—such as one obtained from a subject or patient—comprising contacting the sample with an antigen recognizing construct specifically binding to said TSA peptide, or to the TSA peptide/MHC complex, and detecting the binding between said antigen recognizing construct and said TSA peptide, or to the TSA peptide/MHC complex. In some embodiments, the antigen recognizing construct is a TCR or antibody, or similar constructs, or preferably the antigen recognizing construct according to the herein described invention. In some embodiments, the (biological) sample is a sample of a tumor or a cancer (such as one of those described elsewhere herein) for example a sample comprising tumor or cancer cells.

Also provided is a method of treating cancer in a subject in need thereof, comprising:
a) isolating a cell from said subject;
b) transforming the cell with at least one vector encoding an antigen recognizing construct of the present invention to produce a transformed cell;
c) expanding the transformed cell to produce a plurality of transformed cells; and
d) administering the plurality of transformed cells to said subject.

Also provided is a method of treating cancer in a subject in need thereof, comprising:
a) isolating a cell from a healthy donor;
b) transforming the cell with a vector encoding an antigen recognizing construct of the present invention to produce a transformed cell;
c) expanding the transformed cell to produce a plurality of transformed cells; and
d) administering the plurality of transformed cells to said subject.

Also provided is a method of treating cancer in a subject in need thereof, comprising:
a) isolating a cell from a transgenic mouse bearing unrearranged αβTCR gene loci and human HLA-A*0201;
b) transforming the cell with a vector encoding an antigen recognizing construct of the present invention to produce a transformed cell;
c) expanding the transformed cell to produce a plurality of transformed cells; and
d) administering the plurality of transformed cells to said subject.

Also provided is a method of detecting cancer in a biological sample comprising:
a) contacting the biological sample with an antigen recognizing construct of the present description;
b) detecting binding of the antigen recognizing construct to the biological sample.

In some embodiments, the method of detecting cancer is carried out in vitro, in vivo or in situ.

Also provided is a method of detecting the presence of a condition in a mammal. The method comprises (i) contacting a sample comprising one or more cells from the mammal with any of the inventive TCRs (and functional variants thereof), polypeptides, proteins, nucleic acids, recombinant expression vectors, host cells, populations of cells, antibodies, or antigen binding portions thereof, or pharmaceutical compositions described herein, thereby forming a complex, and detecting the complex, wherein detection of the complex is indicative of the presence of the condition in the mammal, wherein the condition is cancer, such as a TSA expressing malignancy.

With respect to the inventive method of detecting a condition in a mammal, the sample of cells can be a sample comprising whole cells, lysates thereof, or a fraction of the whole cell lysates, e.g., a nuclear or cytoplasmic fraction, a whole protein fraction, or a nucleic acid fraction.

For purposes of the inventive detecting method, the contacting can take place in vitro or in vivo with respect to the mammal. Preferably, the contacting is in vitro.

Also, detection of the complex can occur through any number of ways known in the art. For instance, the inventive antigen recognizing constructs (and functional variants thereof), polypeptides, proteins, nucleic acids, recombinant expression vectors, host cells, populations of cells, or antibodies or TCRs, or antigen binding portions thereof, described herein, can be labeled with a detectable label such as, for instance, a radioisotope, a fluorophore (e.g., fluorescein isothiocyanate (FITC), phycoerythrin (PE)), an enzyme (e.g., alkaline phosphatase, horseradish peroxidase), and element particles (e.g., gold particles).

For purposes of the inventive methods, wherein host cells or populations of cells are administered, the cells can be cells that are allogeneic or autologous to the mammal. Preferably, the cells are autologous to the mammal.

With respect to the above mentioned medical applications of the TCR material of the invention, the to be treated and/or diagnosed cancer can be any cancer, including any of acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vagina, cancer of the vulva, chronic lymphocytic leukemia, chronic myeloid cancer, colon cancer, esophageal cancer, cervical cancer, gastrointestinal carcinoid tumor, glioma, Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, cancer of the oropharynx, ovarian cancer, cancer of the penis, pancreatic cancer, peritoneum, omentum, and mesentery cancer, pharynx cancer, prostate cancer, rectal cancer, renal cancer, skin cancer, small intestine cancer, soft tissue cancer, stomach cancer, testicular cancer, thyroid cancer, cancer of the uterus, ureter cancer, and urinary bladder cancer. A preferred cancer is cancer is cancer of the uterine cervix, oropharynx, anus, anal canal, anorectum, vagina, vulva, or penis. Preferred cancer of the invention are described herein elsewhere.

In general, the invention provides a method for treating a subject suffering from a tumor or tumor disease comprising the administration of the antigen recognizing constructs, nucleic acids, vectors, pharmaceutical compositions and/or host cell as disclosed by the present invention. Preferably the subject is a subject in need of such a treatment. The subject in preferred embodiments is a mammalian subject, preferably a human patient, suffering from a tumor or tumor disease, which is TSA-positive.

In view of the disclosure herein it will be appreciated that the invention furthermore pertains to the following items:

Item 1: An antigen recognizing construct comprising at least one complementary determining region (CDR) 3 having at least 50%, 60%, 70%, 80%, 90%, 95%, 98%, 99%, or 100% sequence identity to an amino acid sequence selected from SEQ ID NOs. 3, 7,11, 15, 19, 23, 27, 31, 35, 39, 43, 47, 51, 55, 59, 63, 67, and 71, in each case independently, optionally with not more than three or two, preferably no more than one, amino acid substitution(s), insertion(s) or deletion(s) compared to these sequences.

Item 2: The antigen recognizing construct according to item 1, wherein said antigen recognizing construct is capable of specifically and/or selectively binding to a TSA of the invention antigenic peptide.

Item 3: The antigen recognizing construct according to item 1 or 2, wherein the antigen recognizing construct is an antibody, or derivative or fragment thereof, or a T cell receptor (TCR), or a derivative or fragment thereof.

Item 4: The antigen recognizing construct according to any one of items 1 to 3, wherein said antigen recognizing construct binds to a human leucocyte antigen (HLA) presented TSA antigenic peptide, wherein said HLA is optionally type A2.

Item 5: The antigen recognizing construct according to any one of items 1 to 4, wherein the construct specifically and/or selectively binds to an epitope having the amino acid sequence selected from SEQ ID NO: 73 to 78, preferably 76, 77 or 78.

Item 6: The antigen recognizing construct according to any one of items 1 to 5, wherein the construct is an α/β-TCR, or fragment or derivative thereof, or the construct is a γ/δ-TCR, or a fragment or derivative thereof.

Item 7: The antigen recognizing construct according to any one of items 1 to 6, characterized in that the construct is of human origin and specifically and/or selectively recognizes a TSA antigenic peptide.

Item 8: The antigen recognizing construct according to any one of items 1 to 7, wherein said antigen recognizing construct is capable of inducing an immune response in a subject, optionally wherein the immune response is characterized by an increase in interferon (IFN) γ levels.

Item 9: The antigen recognizing construct according to any one of items 1 to 8, comprising a TCR α or γ chain; and/or a TCR β or δ chain; wherein the TCR α or γ chain comprises a CDR3 having at least 50%, 60%, 70%, 80%, 90o%, 95%, 98%, 99%, or 100% sequence identity to an amino acid sequence selected from SEQ ID Nos. 3, 11, 19, 27, 35, 43, 51, 59, and 67, and/or wherein the TCR γ or δ chain comprises a CDR3 having at least 50%, 60%, 70%, 80%, 90%, 95%, 98%, 99%, or 100% sequence identity to an amino acid sequence selected from SEQ ID Nos. 7, 15, 23, 31, 39, 47, 55, 63, and 71, in each case independently, optionally with not more than three or two, preferably no more than one, amino acid substitution(s), insertion(s) or deletion(s) compared to these sequences.

Item 10: The antigen recognizing construct according to item 9, wherein the TCR α or γ chain further comprises a CDR1 having at least 50%, 60%, 70%, 80%, 90%, 95%, 98%, 99%, or 100% sequence identity to an amino acid sequence selected from SEQ ID Nos. 1, 9, 17, 25, 33, 41, 49, 57, and 65; and/or a CDR2 having at least 50%, 60%, 70%, 80%, 90%, 95%, 98%, 99%, or 100% sequence identity to an amino acid sequence selected from SEQ ID Nos. 2, 10, 18, 26, 34, 42, 50, 58, and 66, in each case independently, optionally with not more than three or two, preferably no more than one, amino acid substitution(s), insertion(s) or deletion(s) compared to these sequences.

Item 11: The antigen recognizing construct according to item 9 or 10, wherein the TCR β or δ chain further comprises a CDR1 having at least 50%, 60%, 70%, 80%, 90%, 95%, 98%, 99%, or 100% sequence identity to an amino acid sequence selected from SEQ ID Nos. 5, 13, 21, ²9, 37, 45, 53, 61, and 69; and/or a CDR2 having at least 50%, 60%, 70%, 80%, 90%, 95%, 98%, 99%, or 100% sequence identity to an amino acid sequence selected from SEQ ID Nos. 6, 14, 22, 30, 38, 46, 54, 62, and 70, in each case independently, optionally with not more than three or two, preferably no more than one, amino acid substitution(s), insertion(s) or deletion(s) compared to these sequences.

Item 12: The antigen recognizing construct according to any of items 1 to 11, comprising a TCR variable chain region having at least 50%, 60%, 70%, 80%, 90%, 95%, 98%, 99%, or 100% sequence identity to an amino acid sequence selected from SEQ ID Nos. 4, 8, 12, 16, 20, 24, 28, 32, 36, 40, 44, 48, 52, 56, 60, 64, 68, and 72, in each case independently, optionally with not more than three or two, preferably no more than one, amino acid substitution(s), insertion(s) or deletion(s) compared to these sequences.

Item 13: The antigen recognizing construct according to any of items 1 to 12, wherein the construct is humanized, chimerized and/or murinized.

Item 14: The antigen recognizing construct according to any of items 1 to 13, comprising a binding fragment of a TCR, and wherein said binding fragment comprises CDR1 to CDR3 optionally selected from the CDR1 to CDR3 sequences having the amino acid sequences of SEQ ID Nos. 1 to 3, 5 to 7, 9 to 11, 13 to 15, 17 to 19, 21 to 23, 25 to 27, 29 to 31, 33 to 35, 37 to 39, 41 to 43, 45 to 47, 49 to 51, 53 to 55, 57 to 59, 61 to 63, 65 to 67, and 69 to 71, in each case independently, optionally with not more than three or two, preferably no more than one, amino acid substitution(s), insertion(s) or deletion(s) compared to these sequences.

Item 15: The antigen recognizing construct according to any of items 1 to 14, wherein the construct is a TCR, or a fragment thereof, composed of at least one TCR α and one TCR β chain sequence, wherein said TCR α chain sequence and said TCR β chain sequence is selected from the following combinations:

| Alpha Chain CDR1, CDR2, CDR3 | | | Beta Chain CDR1, CDR2, CDR3 | | |
|---|---|---|---|---|---|
| (SEQ ID NO) | | | (SEQ ID NO) | | |
| 1 | 2 | 3 | 5 | 6 | 7 |
| 9 | 10 | 11 | 13 | 14 | 15 |
| 17 | 18 | 19 | 21 | 22 | 23 |
| 25 | 26 | 27 | 29 | 30 | 31 |
| 33 | 34 | 35 | 37 | 38 | 39 |
| 41 | 42 | 43 | 45 | 46 | 47 |
| 49 | 50 | 51 | 53 | 54 | 55 |
| 57 | 58 | 59 | 61 | 62 | 63 |
| 65 | 66 | 67 | 69 | 70 | 71 |

in each case independently, optionally with not more than three or two, preferably no more than one, amino acid substitution(s), insertion(s) or deletion(s) compared to these sequences.

Item 16: The antigen recognizing construct according to any of items 1 to 15, wherein the construct is a TCR, or a fragment thereof, comprising at least one TCR α and one TCR β chain sequence, wherein said TCR α chain sequence and said TCR β chain sequence is selected from the following combinations of alpha and beta chains sequences:

| Alpha Chain Variable | Beta Chain Variable |
|---|---|
| Region (SEQ ID NO:) | Region (SEQ ID NO:) |
| 4 | 8 |
| 12 | 16 |
| 20 | 24 |
| 28 | 32 |
| 36 | 40 |
| 44 | 48 |
| 52 | 56 |
| 60 | 64 |
| 68 | 72 |

in each case independently, optionally with not more than three or two, preferably no more than one, amino acid substitution(s), insertion(s) or deletion(s) compared to these sequences.

Item 17: The antigen recognizing construct according to any of items 1 to 16, wherein the construct is a TCR, or a fragment thereof, further comprising a TCR constant region preferably a human or mouse TCR constant region.

Item 18: A nucleic acid encoding for an antigen recognizing construct according to any one of items 1 to 17.

Item 19: A vector comprising a nucleic acid according to item 18.

Item 20: A host cell comprising an antigen recognizing construct according to any one of items 1 to 17, or a nucleic acid according to item 18, or a vector according to item 19.

Item 21: The host cell according to item 20, wherein the cell is a lymphocyte, preferably a T lymphocyte or T lymphocyte progenitor, more preferably a CD4 or CD8 positive T-cell.

Item 22: A pharmaceutical composition comprising the antigen recognizing construct according to any of items 1 to 17, or the nucleic acid according to item 18, or the vector according to item 19, or the host cell according to item 20 or 21, and a pharmaceutical acceptable carrier, stabilizer and/or excipient.

Item 23: The antigen recognizing construct according to any one of items 1 to 17, or a nucleic acid according to item 18, or a vector according to item 19, or a host cell according to item 20 or 21, or the pharmaceutical composition according to item 22, for use in medicine.

Item 24: The antigen recognizing construct, or the nucleic acid, or the vector, or the host cell, or the pharmaceutical composition, for use according to item 23, for use in the diagnosis, prevention, and/or treatment of a proliferative disease, wherein the disease comprises a malignant or benign tumor disease.

Item 25: The antigen recognizing construct, or the nucleic acid, or the vector, or the host cell, or the pharmaceutical composition, for use according to item 24 wherein the tumor disease is characterized by the expression of TAA or TSA in a tumor cell of the tumor disease.

Item 26: The antigen recognizing construct, or the nucleic acid, or the vector, or the host cell, or the pharmaceutical composition, for use according to any one of items 23 to 25, wherein the use in medicine is a use in immune therapy optionally comprising an adoptive cell transfer, wherein the immune therapy comprises adoptive autologous or heterologous T-cell therapy.

Item 27: A method of manufacturing a TSA specific antigen recognizing construct expressing cell line, comprising

a. providing a suitable host cell,

b. providing a genetic construct comprising a coding sequence encoding the antigen recognizing construct according to any of items 1 to 17,

c. introducing into said suitable host cell said genetic construct,

d. expressing said genetic construct by said suitable host cell.

Item 28: The method according to item 27, further comprising cell surface presentation of said antigen recognizing construct.

Item 29: The method according to item 27 or 28, wherein the genetic construct is an expression construct comprising a promoter sequence operably linked to said coding sequence.

Item 30: The method according to any one of items 27 to 29, wherein said antigen recognizing construct is of mammalian origin, preferably of human origin.

Item 31: The method according to any one of items 27 to 30, wherein said suitable host cell is a mammalian cell, optionally selected from a human cell or a human T lymphocyte.

Item 32: The method according to any of items 27 to 31, wherein said antigen recognizing construct is a modified TCR, wherein said modification comprises addition of a functional domain comprising a label, or an alternative domain comprising a membrane anchor domain.

Item 33: The method according to item 32, wherein said antigen recognizing construct is an alpha/beta TCR, gamma/delta TCR, or a single chain TCR (scTCR).

Item 34: The method according to any of items 27 to 33, wherein said genetic construct is introduced into said suitable host cell by retroviral transfection.

Item 35: The method according to any of items 27 to 34, further comprising the isolation and purification of the antigen recognizing construct from the suitable host cell and, optionally, reconstitution of the antigen recognizing construct in a T-cell.

The invention further pertains in preferred embodiments to the following items-B:

Item B1. An antigen recognizing construct, comprising at least one complementary determining region which specifically recognizes a mutated Ras antigen such as a mutated RasG12, preferably wherein the spliced peptide variant comprises a sequence according to any one of SEQ ID NO: 76 to 78.

Item B2. The antigen recognizing construct according to item B 1 comprising at least one complementary determining region (CDR) 3 having at least 80% sequence identity to an amino acid sequence selected from SEQ ID NOs. 3, 7, 11, 15, 19, 23, 27, 31, 35, 39, 43, 47, 51, 55, 59, 63, 67, and 71, in each case independently, optionally with not more than three or two, preferably no more than one, amino acid substitution(s), insertion(s) or deletion(s) compared to these sequences.

Item B3. The antigen recognizing construct according to item B 1 or 2, wherein the antigen recognizing construct is an α/β-TCR, or fragment or derivative thereof, or the construct is a γ/δ-TCR, or a fragment or derivative thereof.

Item B4. The antigen recognizing construct according to any one of item Bs 1 to 3, comprising a TCR α or γ chain; and/or a TCR β or δ chain; wherein the TCR α or γ chain comprises a CDR3 having at least 80% sequence identity to an amino acid sequence selected from SEQ ID Nos. 3, 11, 19, 27, 35, 43, 51, 59, and 67, and/or wherein the TCR β or δ chain comprises a CDR3 having at least 80% sequence identity to an amino acid sequence selected from SEQ ID Nos. 7, 15, 23, 31, 39, 47, 55, 63, and 71; in each case independently, optionally with not more than three or two, preferably no more than one, amino acid substitution(s), insertion(s) or deletion(s) compared to these sequences.

Item B5. The antigen recognizing construct according to any of item Bs 1 to 4, comprising a TCR variable chain region having at least 80% sequence identity to an amino acid sequence selected from SEQ ID Nos. 4, 8, 12, 16, 20, 24, 28, 32, 36, 40, 44, 48, 52, 56, 60, 64, 68, and 72, in each case independently, optionally with not more than three or two, preferably no more than one, amino acid substitution(s), insertion(s) or deletion(s) compared to these sequences.

Item B6. The antigen recognizing construct according to any of item Bs 1 to 5, wherein the construct is fully or partially humanized, chimerized and/or murinized.

Item B7. The antigen recognizing construct according to any of item Bs 1 to 6, wherein the construct is a TCR, or a fragment thereof, composed of at least one TCR α and one TCR β chain sequence, wherein said TCR α chain sequence and said TCR β chain sequence is selected from the following combinations:

in each case independently, optionally with not more than three or two, preferably no more than one, amino acid substitution(s), insertion(s) or deletion(s) compared to these sequences.

Item B8. The antigen recognizing construct according to any of item Bs 1 to 7, wherein the construct is a TCR, or a fragment thereof, further comprising a TCR constant region preferably a human or mouse TCR constant region.

Item B9. A nucleic acid encoding for an antigen recognizing construct according to any one of item Bs 1 to 8.

Item B10. A vector comprising a nucleic acid according to item B 18.

Item B11. A host cell comprising an antigen recognizing construct according to any one of item Bs 1 to 8, or a nucleic acid according to item B 9, or a vector according to item B 10.

Item B12. A pharmaceutical composition comprising the antigen recognizing construct according to any of item Bs 1 to 8, or the nucleic acid according to item B 9, or the vector according to item B 10, or the host cell according to item B11, and a pharmaceutical acceptable carrier, stabilizer and/or excipient.

Item B13. A composition for use in medicine, wherein the composition comprises the antigen recognizing construct according to any one of item Bs 1 to 8, or the nucleic acid according to item B 9, or the vector according to item B 10, or the host cell according to item B 11, or the pharmaceutical composition according to item B 12.

Item B14. The composition for use according to item B 13, for use in the diagnosis, prevention, and/or treatment of a proliferative disease, wherein the disease comprises a malignant or benign tumor disease, preferably a tumor disease which is positive for the RasG12V mutation.

Item B15. The composition for use according to item B 13 or 14, wherein the use in medicine is a use in immune therapy optionally comprising an adoptive cell transfer, wherein the immune therapy comprises adoptive autologous or heterologous T-cell therapy.

16. A method of manufacturing a TSA specific antigen recognizing construct expressing cell line, comprising

a. providing a suitable host cell,

b. providing a genetic construct comprising a coding sequence encoding the antigen recognizing construct according to any of item Bs 1 to 8,

c. introducing into said suitable host cell said genetic construct,

d. expressing said genetic construct by said suitable host cell.

The present invention will now be further described in the following examples with reference to the accompanying figures and sequences, nevertheless, without being limited thereto. For the purposes of the present invention, all references as cited herein are incorporated by reference in their entireties. In the Figures and Sequences:

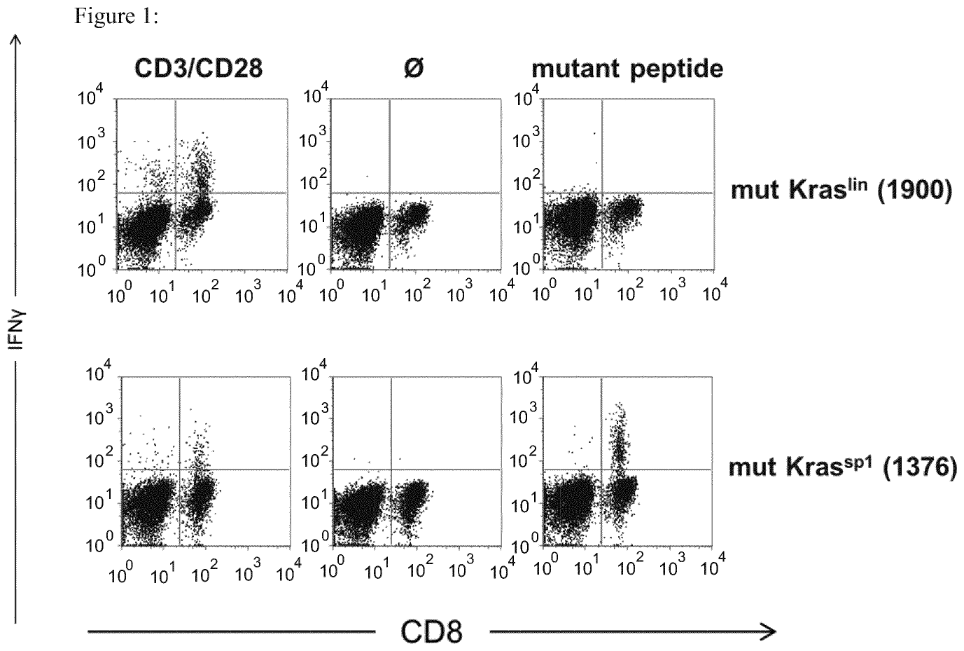

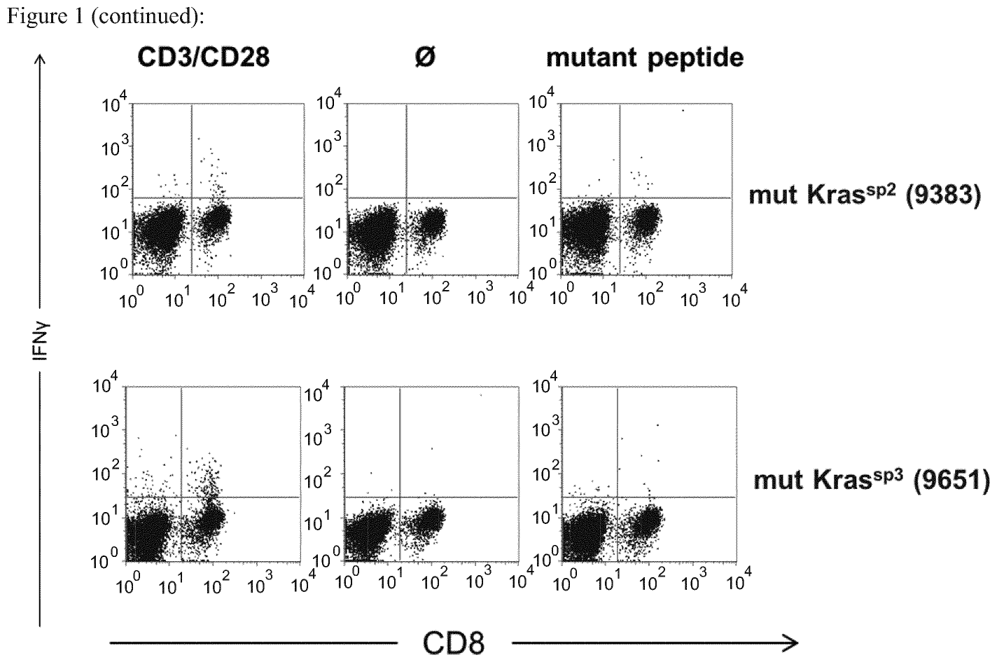

FIG. 1: Generation of TCRs specific for spliced epitopes of mutant Kras^{G12V}. Kras harbors the oncogenic mutation G12V in approximately 30% of pancreatic ductal adeno carcinoma and 20% of the colon and non-small cell lung cancers. Representative examples of ex vivo ICS analysis of Kras mutant peptide immunized ABabDII mice (Li et al Nat Med 2010) 7 days after the last immunization, linear as well as spliced epitopes 1-3 were used. Stimulation with CD3/CD28 beads served as positive control, stimulation without peptide (Ø) was used as negative control. Numbers in brackets represent percent IFNγ⁺ CD8⁺ T cells, respectively. Lin: linear epitope, KLVVVGAVGV; sp1: spliced epitope 1, KLVVGAVGV; sp2: spliced epitope 2: KLVVVAVGV; sp3: spliced epitope 3 YLVVVGAVGV. Spleens of mice with IFNγ-reactive CD8+ T cells were cultured for 10 days in the presence of 10⁻⁸ M of the respective mutant Kras peptide and mutation-specific CD8⁺ T cells were purified by IFNγ-capture assay for isolation of TCR α and β chains by RACE-PCR.

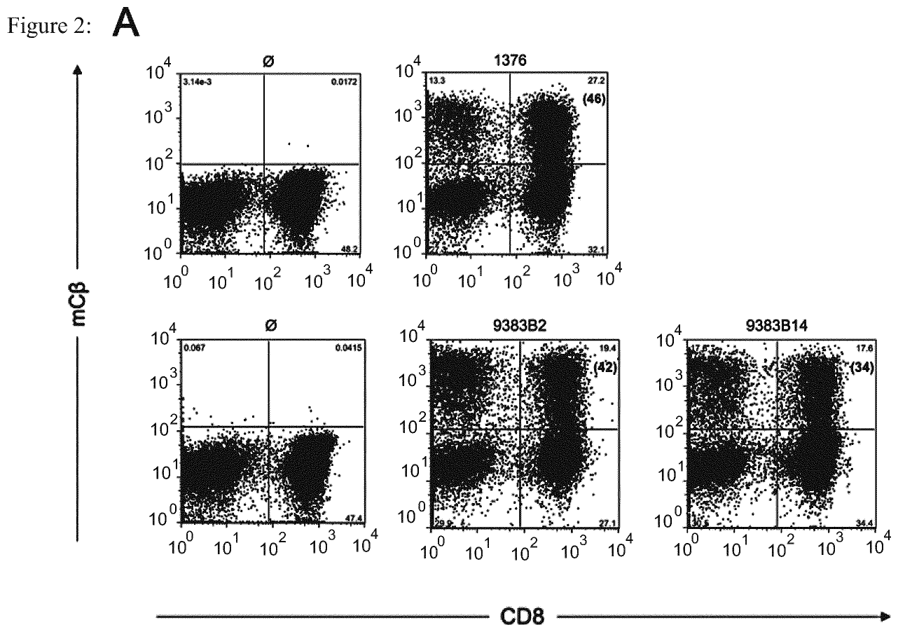

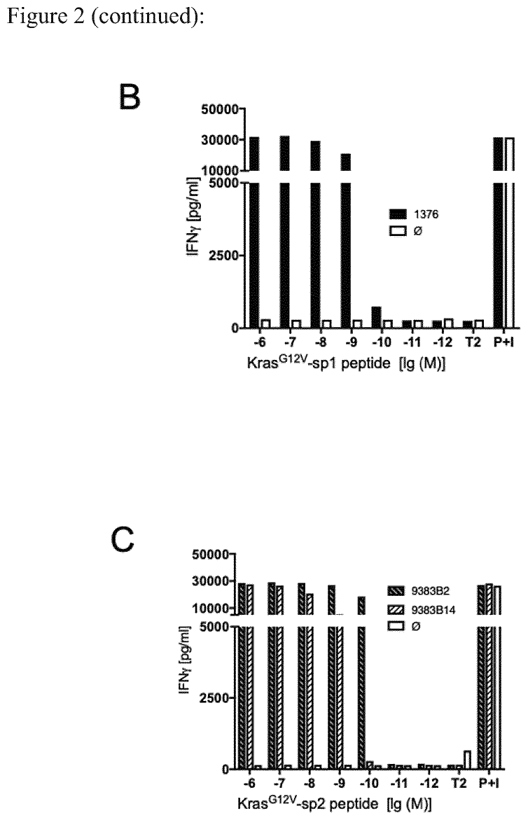

FIG. 2: TCR gene transfer confers specificity for mutant spliced Kras^{G12V} peptides KLVVGAVGV (sp¹) and KLVVVAVGV (sp²). The corresponding TCR α and β chains isolated from one Kras^{G12V} sp1 and one Kras^{G12V} sp2 peptide immunized ABabDII mouse, respectively (1376 and 9383B2/B14), were cloned into retroviral vector pMP71 and reexpressed in human PBMC. (A) Transduction efficacy was measured by staining of the mouse TCRβ chain on CD8⁺ T cells, number of positive CD8⁺ T cells is shown in brackets. (B and C) IFNγ-production of Kras^{G12V} TCR-transduced T cells upon coculture with peptide-loaded T2 cells. Either mutant splice epitope 1 (B, 1376, solid bars) or mutant splice epitope 2 [C, 9383B2 (hatched bars) and 9383B14 (open hatched bars)] peptide was used for loading; as negative control T2 cells were not loaded, for maximal stimulation PMA and ionomycin (P+I) were added to the coculture. All target cells were also cocultured with non-transduced T cells (Ø, open bars).

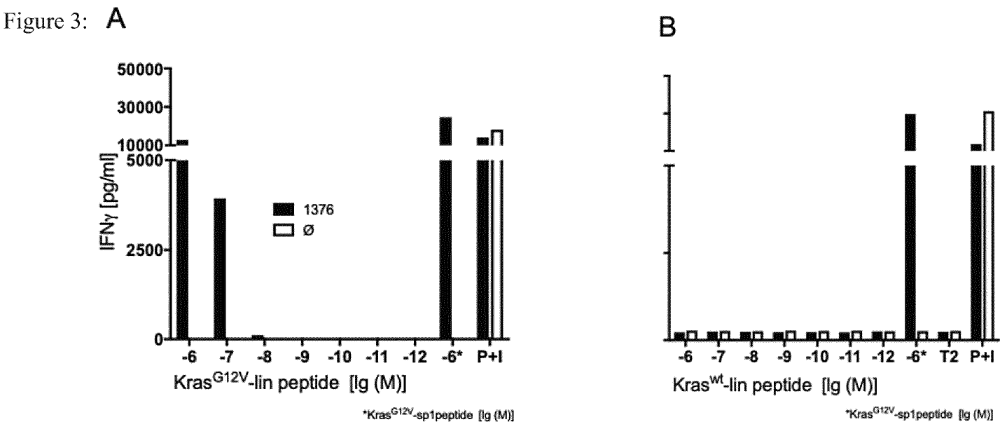

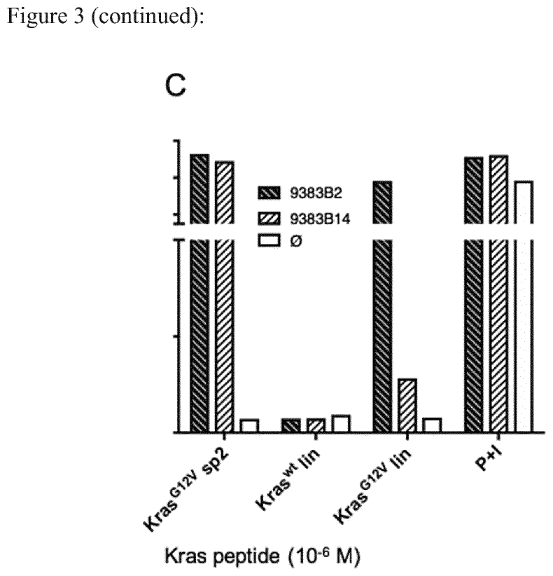

FIG. 3: TCRs specific for spliced epitopes cross-react with linear mutant but not wildtype Kras peptide. IFNγ-production of Kras^{G12V} 1376 and 9383B2/B14 TCR-transduced T cells upon coculture with peptide-loaded T2 cells. For maximal stimulation PMA and ionomycin (P+I) were added to the coculture, all target cells were also cocultured with non-transduced T cells (Ø, open bars). (A) IFNγ-production of Kras^{G12V} TCR₁₃₇₆-transduced T cells upon coculture with T2 cells loaded with linear Kras^{G12V} peptide epitope (KLVVVGAVGV) (solid bars). (B) IFNγ-production of Kras^{G12V} TCR₁₃₇₆-transduced T cells upon coculture with T2 cells loaded with linear Kras^{wt} peptide epitope (KLVVVGAGGV)(solid bars). As positive control in A and B, IFNγ-production of Kras^{G12V} TCR₁₃₇₆-transduced T cells upon coculture with T2 cells loaded with spliced Kras^{G12V} peptide epitope (10⁻⁶ M, −6*) was used. (C) IFNγ-production of mutant splice epitope 2 specific TCRs 9383B2 (hatched bars) and 9383B14 (open hatched bars) were tested on T2 cells loaded with the respective splice epitope (Kras^{G12V} sp2, KLVVVAVGV) or linear wildtype (Kras^{wt} lin) and mutant (Kras^{G12V} lin) peptide.

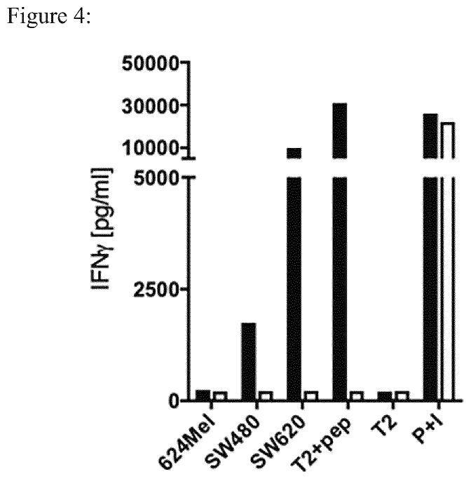

FIG. 4: The Kras^{G12V} epitope is processed and recognized by TCR-redirected T cells. For analysis of natural processing and recognition of Kras^{G12V} epitopes, Kras^{G12V} colon carcinoma cell lines SW480 and SW620 and Kras^{wt} melanoma cell line 624Mel were cocultured with Kras^{G12V}TCR₁₃₇₆-redirected T cells, IFNγ-production of transduced T cells is shown (solid bars). As positive control peptide loaded T2 cells were used, non-loaded T2 cells served as negative control. For maximal stimulation PMA and ionomycin (P+I) were added, all target cells were also cocultured with non-transduced T cells (open bars).

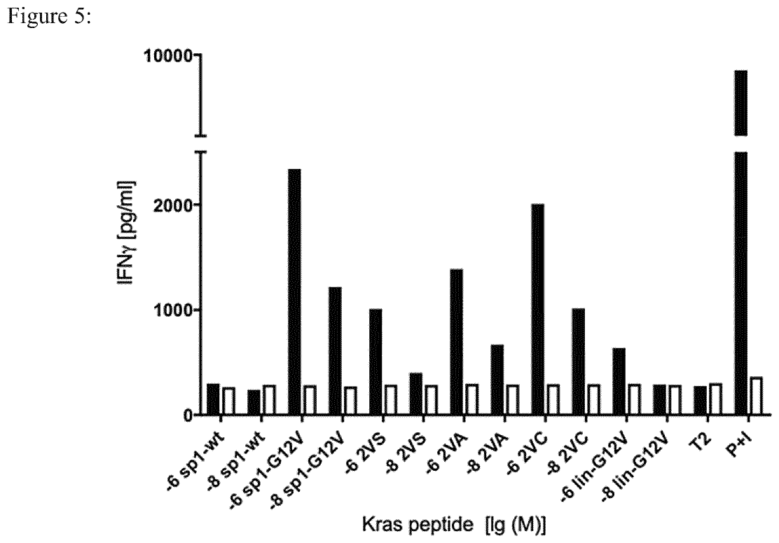

FIG. 5: TCR gene transfer confers cross-recognition for spliced Kras peptide with G12S, A and C substitution. IFNγ-production of Kras^{G12V} TCR₁₃₇₆-transduced T cells (red bars) upon coculture with peptide-loaded T2 cells (10⁻⁶ and 10⁻⁸ M); as negative control T2 cells were not loaded, for maximal stimulation PMA and ionomycin (P+I) were added. Cells were also cocultured with non-transduced T cells (grey bars). Nomenclature for peptide epitopes is as follows: sp1-wt (KLVVGAGGV), sp1-G12V (KLVVGAVGV), 2VS (KLVVGASGV), 2VA (KLVVGAAGV), 2VC (KLVVGACGV) and lin-G12V (KLVVVGAVGV).

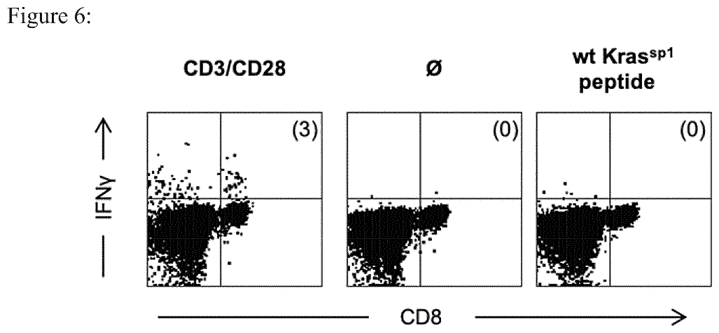

FIG. 6: The spliced Kras^{wt} peptide epitope is not immunogenic in ABabDII mice. Representative examples of ex vivo ICS analysis of Kras spliced wildtype peptide (wt Kras^{sp1}, KLVVGAGGV) immunized ABabDII mice (n=3) seven days after the 4th immunization. Stimulation with CD3/CD28 beads served as positive control, stimulation without peptide (Ø) was used as negative control. Numbers in brackets represent percent IFNγ+ CD8⁺ T cells, respectively.

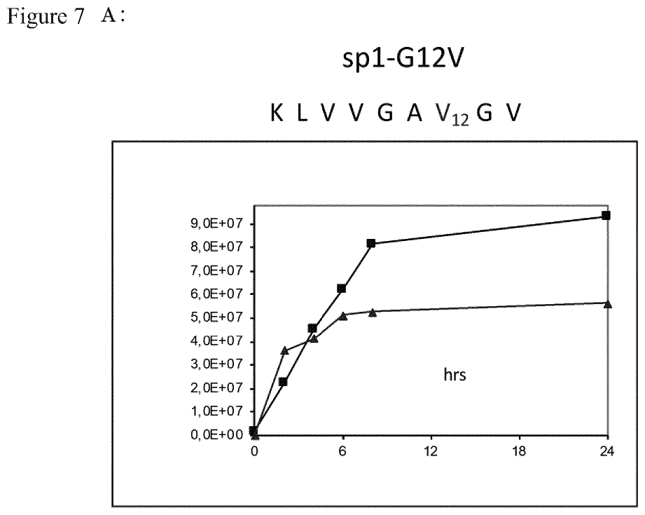

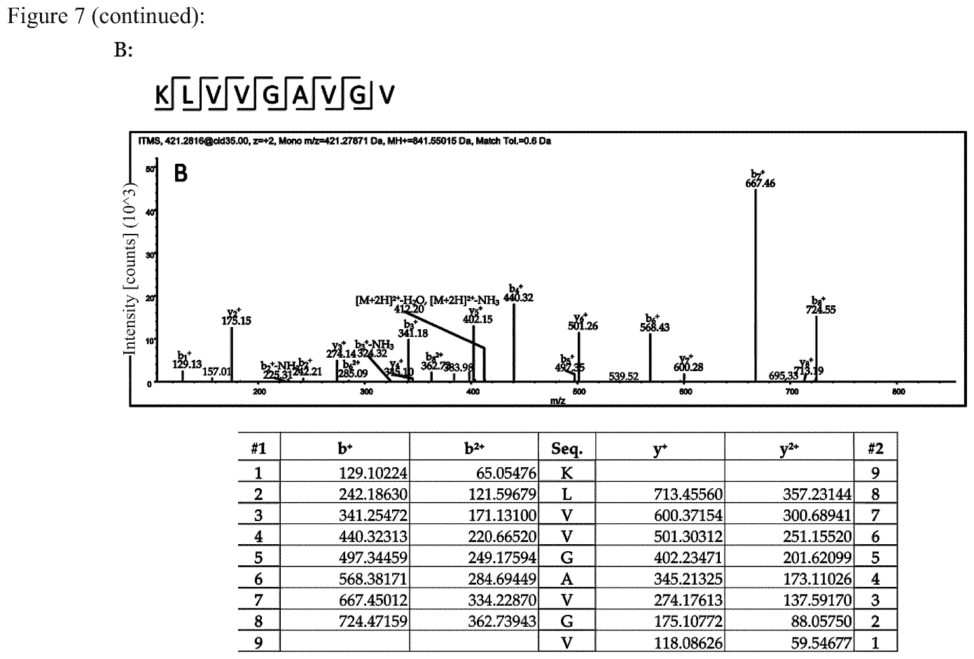

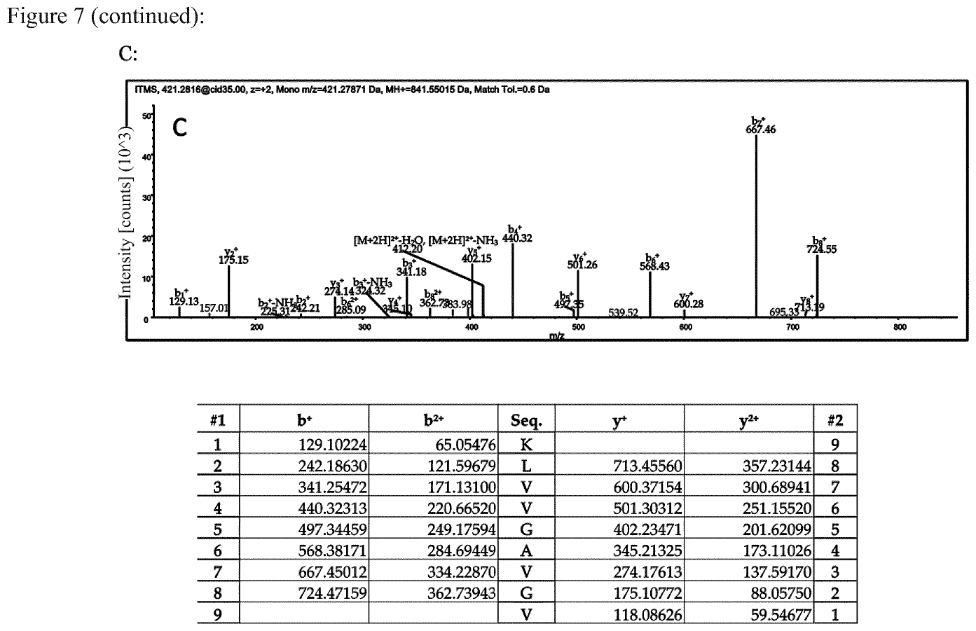

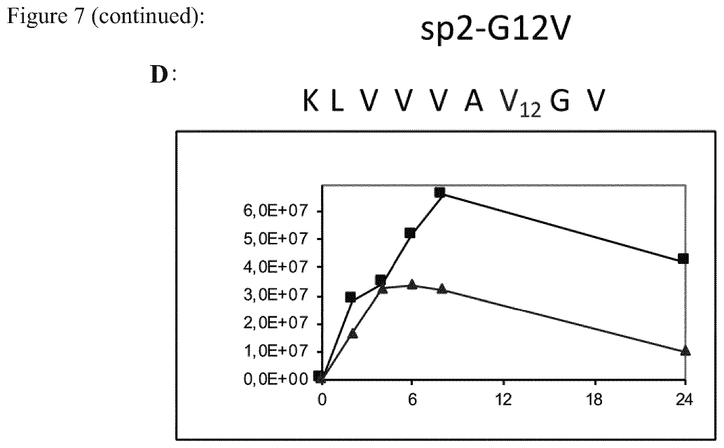

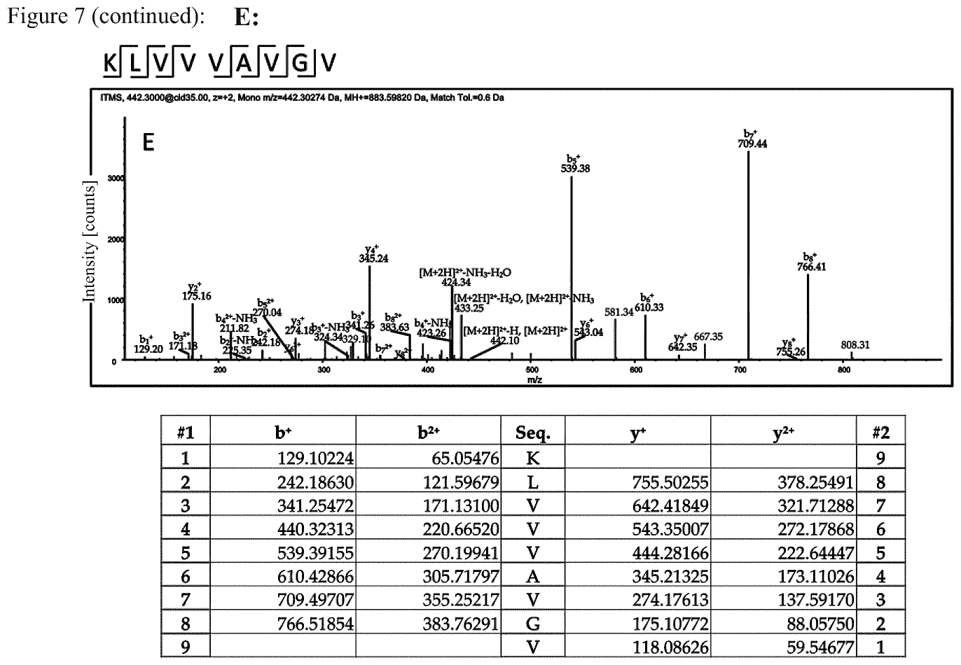

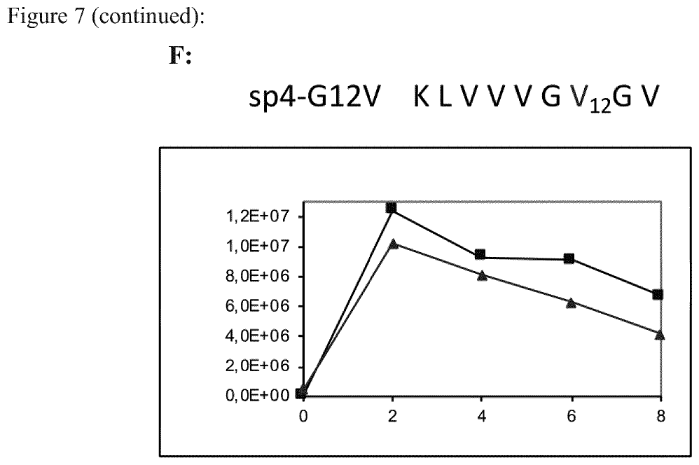

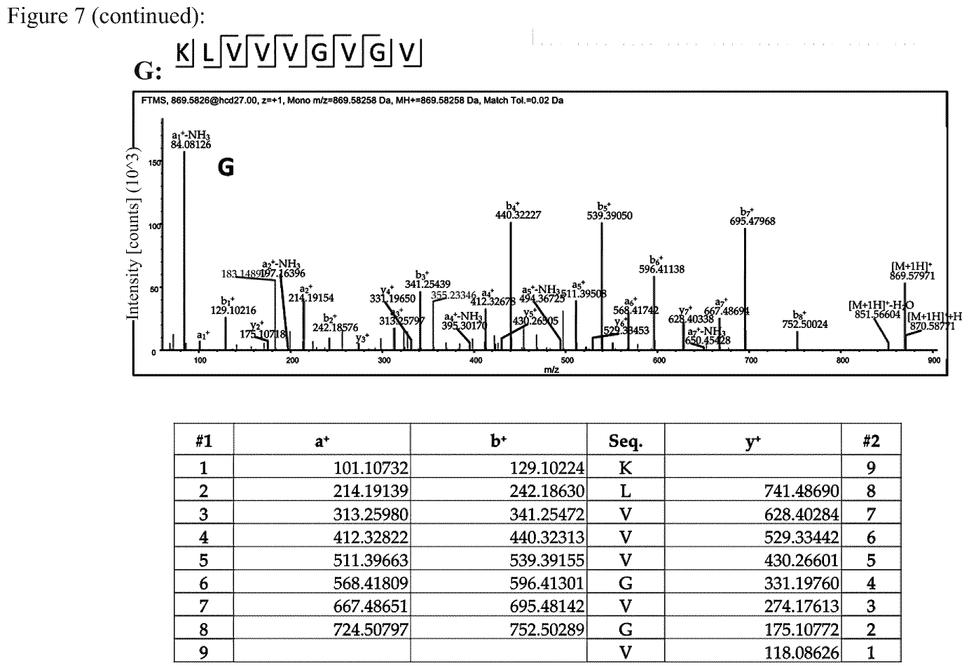

FIG. 7: Kinetic analysis of peptides A: kinetics of in vitro sp1-G12V spliced peptide KLVVGAVGV; B and C: Comparison of the MSMS spectra of the peptide KLVVGAVGV generated by proteasome (B) or synthetically (C); C: D: generation kinetics of KLVVVAVGV; E: MSMS spectra of proteasomal generated KLVVVAVGV; F: kinetics of the spliced peptide KLVVVGVGV; G: spectrum of predicted KRAS peptide KLVVVGVGV.

TABLE 1
TCR sequences of the invention

| SEQ | | | | |
|---|---|---|---|---|
| ID | | | | |
| NO: | TCR | Chain | Region | Sequence |
| 1 | 1376a | alpha | CDR1 | DSSSTY |
| | TRAV5*01 F | | | |
| | TRAJ24*02 F | | | |
| 2 | | alpha | CDR2 | IFSNMDM |
| 3 | | alpha | CDR3 | CAESTDSWGKLQF |
| 4 | | alpha | Variable | MKTFAGFSF LFLWLQLDCM |
| | | | | SRGEDVEQSLFLSVREGDSS VINCTYTDSS |
| | | | | STYLYWYKQE PGAGLQLLTY |
| | | | | IFSNMDMKQD |
| | | | | QRLTVLLNKKDKHLSLRIAD TQTGDSAIYF |
| | | | | CAESTDSWGK LQFGAGTQVV VTPD |
| 5 | 137613 | beta | CDR1 | MGHRA |
| | TRBV4-1*01 F | | | |
| | TRBJ2-7*01 F | | | |
| | TRBD2*01 F | | | |
| 6 | | beta | CDR2 | YSYEKL |
| 7 | | beta | CDR3 | CASSQDLAGYEQYF |
| 8 | | beta | Variable | MGCRLL CCAVLCLLGA VPIDTEVTQT |
| | | | | PKHLVMGMTN KKSLKCEQHM |
| | | | | GHRAMYWYKQKAKKPPELMF |
| | | | | VYSYEKLSIN ESVPSRFSPE CPNSSLLNLH |
| | | | | LHALQPEDSA LYLCASSQDLAGYEQYFGPG |
| | | | | TRLTVT |
| 9 | 1377a | alpha | CDR1 | NSAFQY |
| | TRAV12-3*01 | | | |
| | F TRAJ58*01 | | | |
| 10 | | alpha | CDR2 | TYSSGN |
| 11 | | alpha | CDR3 | CAIFSGSRLTF |
| 12 | | alpha | Variable | MMKSLRVLLVILWLQLSWVWSQQKEVEQD |
| | | | | PGPLSVPEGAIVSLNCTYSNSAFQYFMVVYR |
| | | | | QYSRKGPELLMYTYSSGNKEDGRFTAQVDK |
| | | | | SSKYISLFIRDSQPSDSATYLCAIFSGSRLTFG |
| | | | | EGTQLTVNPD |
| 13 | 1377b | beta | CDR1 | SQVTM |
| | TRBV29-1* | | | |
| | 01FTRBJ2-1* | | | |
| | 01FTRBD2* | | | |
| | 01F | | | |
| 14 | | beta | CDR2 | ANQGSEA |
| 15 | | beta | CDR3 | CSVAGLAGSSYNEQFF |
| 16 | | beta | Variable | MLSLLLLLLGLGSVFSAVISQKPSRDICQRG |
| | | | | TSLTIQCQVDSQVTMMFWYRQQPGQSLTLI |
| | | | | ATANQGSEATYESGFVIDKFPISRPNLTFSTL |
| | | | | TVSNMSPEDSSIYLCSVAGLAGSSYNEQFFG |
| | | | | PGTRLTVL |
| 17 | 1378a | alpha | CDR1 | TTSDR |
| | TRAV39*01F | | | |
| | TRAJ54*01F | | | |
| 18 | | alpha | CDR2 | LLSNGAV |
| 19 | | alpha | CDR3 | CAGIQGAQKLVF |
| 20 | | alpha | Variable | MKKLLAMILWLQLDRLSGELKVEQNPLFLS |
| | | | | MQEGKNYTIYCNYSTTSDRLYVVYRQDPGKS |
| | | | | LESLFVLLSNGAVKQEGRLMASLDTKARLS |
| | | | | TLHITAAVHDLSATYFCAGIQGAQKLVFGQ |
| | | | | GTRLTINPN |
| 21 | 1378b | beta | CDR1 | MNHEY |
| | TRBV27* | | | |
| | 01FTRBJ2-1* | | | |
| | 01FTRBD2* | | | |
| | 01F | | | |
| 22 | | beta | CDR2 | SMNVEV |
| 23 | | beta | CDR3 | CASSLWTNNEQFF |
| 24 | | beta | Variable | MGPQLLGYVVLCLLGAGPLEAQVTQNPRYL |
| | | | | ITVTGKKLTVTCSQNMNHEYMSWYRQDPG |
| | | | | LGLRQIYYSMNVEVTDKGDVPEGYKVSRKE |
| | | | | KRNFPLILESPSPNQTSLYFCASSLWTNNEQ |
| | | | | FFGPGTRLTVL |
| 25 | 9283a | alpha | CDR1 | TSINN |
| | TRAVtr* | | | |
| | 01FTRAJ49* | | | |
| | 01F | | | |
| 26 | | alpha | CDR2 | IRSNERE |
| 27 | | alpha | CDR3 | CATDEDTGNQFYF |
| 28 | | alpha | Variable | METLLGVSLVILWLQLARVNSQQGEEDPQA |
| | | | | LSIQEGENATMNCSYKTSINNLQVVYRQNSG |
| | | | | RGLVHLILIRSNEREKHSGRLRVTLDTSKKS |
| | | | | SSLLITASRAADTASYFCATDEDTGNQFYFG |
| | | | | TGTSLTVIPN |
| 29 | 9283b2 | beta | CDR1 | SGHNS |
| | TRBV12-3* | | | |
| | 01FTRBJ2-7* | | | |
| | 01FTRBD1* | | | |
| | 01f | | | |
| 30 | | beta | CDR2 | FNNNVP |
| 31 | | beta | CDR3 | CASSLWGYEQYF |
| 32 | | beta | Variable | MDSWTFCCVSLCILVAKHTDAGVIQSPRHE |
| | | | | VTEMGQEVTLRCKPISGHNSLFWYRQTMM |
| | | | | RGLELLIYFNNNVPIDDSGMPEDRFSAKMP |
| | | | | NASFSTLKIQPSEPRDSAVYFCASSLWGYEQ |
| | | | | YFGPGTRLTVT |
| 33 | 9283a | alpha | CDR1 | TSINN |
| | TRAV17* | | | |
| | 01FTRAJ49* | | | |
| | 01F | | | |
| 34 | | alpha | CDR2 | IRSNERE |
| 35 | | alpha | CDR3 | CATDEDTGNQFYF |
| 36 | | alpha | Variable | METLLGVSLVILWLQLARVNSQQGEEDPQA |
| | | | | LSIQEGENATMNCSYKTSINNLQVVYRQNSG |
| | | | | RGLVHLILIRSNEREKHSGRLRVTLDTSKKS |
| | | | | SSLLITASRAADTASYFCATDEDTGNQFYFG |
| | | | | TGTSLTVIPN |
| 37 | 9283b14 | beta | CDR1 | SGHNS |
| | TRBV12-3* | | | |
| | 01FTRBJ2-7* | | | |
| | 01FTRBD1* | | | |
| | 01F | | | |
| 38 | | beta | CDR2 | FNNNVP |
| 39 | | beta | CDR3 | CASSLVGYEQYF |
| 40 | | beta | Variable | MDSWTFCCVSLCILVAKHTDAGVIQSPRHE |
| | | | | VTEMGQEVTLRCKPISGHNSLFWYRQTMM |
| | | | | RGLELLIYFNNNVPIDDSGMPEDRFSAKMP |
| | | | | NASFSTLKIQPSEPRDSAVYFCASSLVGYEQY |
| | | | | FGPGTRLTVT |
| 41 | 9386a | alpha | CDR1 | TSESDYY |
| | TRAV38-2/ | | | |
| | DV8* | | | |
| | 01FTRAJ53* | | | |
| | 01F | | | |
| 42 | | alpha | CDR2 | QEAYKQQN |
| 43 | | alpha | CDR3 | CAFGGSNYKLTF |
| 44 | | alpha | Variable | MACPGFLWALVISTCLEFSMAQTVTQSQPE |
| | | | | MSVQEAETVTLSCTYDTSESDYYLFWYKQP |
| | | | | PSRQMILVIRQEAYKQQNATENRFSVNFQK |
| | | | | AAKSFSLKISDSQLGDAAMYFCAFGGSNYKL |
| | | | | TFGKGTLLTVNPN |
| 45 | 9386b3 | beta | CDR1 | LNHNV |
| | TRBV15*02 | | | |
| | (F)TRBJ2-4* | | | |
| | 01FTRBD2* | | | |
| | 02F | | | |
| 46 | | beta | CDR2 | YYDKDF |
| 47 | | beta | CDR3 | CATSGSQNIQYF |
| 48 | | beta | Variable | MGPGLLHVVMALCLLGTGHGDAMVIQNPR |
| | | | | YQVTQFGKPVTLSCSQTLNHNVMYVVYQQK |
| | | | | SSQAPKLLFHYYDKDFNNEADTPDNFSRR |
| | | | | PNTSFCFLDIRSPGLGDAAMYLCATSGSQNI |
| | | | | QYFGAGTRLSVL |
| 49 | 9386a | alpha | CDR1 | TSESDYY |
| | TRAV38-2/ | | | |
| | DV8* | | | |
| | 01FTRAJ53* | | | |
| | 01F | | | |
| 50 | | alpha | CDR2 | QEAYKQQN |
| 51 | | alpha | CDR3 | CAFGGSNYKLTF |
| 52 | | alpha | Variable | MACPGFLWALVISTCLEFSMAQTVTQSQPE |
| | | | | MSVQEAETVTLSCTYDTSESDYYLFWYKQP |
| | | | | PSRQMILVIRQEAYKQQNATENRFSVNFQK |
| | | | | AAKSFSLKISDSQLGDAAMYFCAFGGSNYKL |
| | | | | TFGKGTLLTVNPN |
| 53 | 9386b12 | beta | CDR1 | MDHEN |
| | TRBV28*01F | | | |
| | TRBJ2-1* | | | |
| | 01FTRBD2* | | | |
| | 01F | | | |
| 54 | | beta | CDR2 | SYDVKM |
| 55 | | beta | CDR3 | CASSQGLALEQFF |
| 56 | | beta | Variable | MGIRLLCRVAFCFLAVGLVDVKVTQSSRYLV |
| | | | | KRTGEKVFLECVQDMDHENMFWYRQDPG |
| | | | | LGLRLIYFSYDVKMKEKGDIPEGYSVSREKK |
| | | | | ERFSLILESASTNQTSMYLCASSQGLALEQF |
| | | | | FGPGTRLTVL |
| 57 | 9651a | alpha | CDR1 | SSVSVY |
| | TRAV8-6* | | | |
| | 01FTRAJ53* | | | |
| | 01F | | | |
| 58 | | alpha | CDR2 | YLSGSTLV |
| 59 | | alpha | CDR3 | CAVSGGSNYKLTF |
| 60 | | alpha | Variable | MLLLLVPAFQVIFTLGGTRAQSVTQLDSQVP |
| | | | | VFEEAPVELRCNYSSSVSVYLFWYVQYPNQ |
| | | | | GLQLLLKYLSGSTLVESINGFEAEFNKSQTS |
| | | | | FHLRKPSVHISDTAEYFCAVSGGSNYKLTFG |
| | | | | KGTLLTVNPN |
| 61 | 9651b | beta | CDR1 | LGHNA |
| | TRBV4-2* | | | |
| | 01FTRBJ2-1* | | | |
| | 01FTRBD2* | | | |
| | 02F | | | |
| 62 | | beta | CDR2 | YNFKEQ |
| 63 | | beta | CDR3 | CASSQESGNLYNEQFF |
| 64 | | beta | Variable | MGCRLLCCAVLCLLGAVPMETGVTQTPRHL |
| | | | | VMGMTNKKSLKCEQHLGHNAMYWYKQSA |
| | | | | KKPLELMFVYNFKEQTENNSVPSRFSPECP |
| | | | | NSSHLFLHLHTLQPEDSALYLCASSQESGNL |
| | | | | YNEQFFGPGTRLTVL |
| 65 | 9652a | alpha | CDR1 | NSMFDY |
| | TRAV29/ | | | |
| | DV5* | | | |
| | 01FTRAJ49* | | | |
| | 01F | | | |
| 66 | | alpha | CDR2 | ISSIKDK |
| 67 | | alpha | CDR3 | CAASRVVDTGNQFYF |
| 68 | | alpha | Variable | MAMLLGASVLILWLQPDWVNSQQKNDDQ |
| | | | | QVKQNSPSLSVQEGRISILNCDYTNSMFDYF |
| | | | | LWYKKYPAEGPTFLISISSIKDKNEDGRFTV |
| | | | | FLNKSAKHLSLHIVPSQPGDSAVYFCAASR |
| | | | | WDTGNQFYFGTGTSLTVIPN |
| 69 | 9652b | beta | CDR1 | MDHEN |
| | TRBV28* | | | |
| | 01FTRBJ2-1* | | | |
| | 01FTRBD2* | | | |
| | 01F | | | |
| 70 | | beta | CDR2 | SYDVKM |
| 71 | | beta | CDR3 | CASSWTSGYNEQFF |
| 72 | | beta | Variable | MGIRLLCRVAFCFLAVGLVDVKVTQSSRYLV |
| | | | | KRTGEKVFLECVQDMDHENMFWYRQDPG |
| | | | | LGLRLIYFSYDVKMKEKGDIPEGYSVSREKK |
| | | | | ERFSLILESASTNQTSMYLCASSWTSGYNEQ |
| | | | | FFGPGTRLTVL |

TABLE 1a

| SEQ ID | | | | |
|---|---|---|---|---|
| NO: | TCR | Chain | Region | Sequence |
| 79 | mod 1376b | beta | CDR1 | LGHRA |
| 80 | | beta | CDR2 | YSYEKL |
| 81 | | beta | CDR3 | CASSQDLAGYEQYF |
| 82 | | beta | Variable | MGCRLL CCAVLCLLGA VPIDTEVTQT |
| | | | | PKHLVMGMTN KKSLKCEQHL |
| | | | | GHRAMYWYKQKAKKPPELMF |
| | | | | VYSYEKLSIN ESVPSRFSPE |
| | | | | CPNSSLLNLH LHALQPEDSA |
| | | | | LYLCASSQDLAGYEQYFGPG TRLTVT |
| 83 | 1377b | beta | CDR1 | LNHNV |
| | TRBV15*01 | | | |
| | F TRBJ2- | | | |
| | 2*01 F | | | |
| | TRBD2*01 F | | | |
| 84 | | beta | CDR2 | YYDKDF |
| 85 | | beta | CDR3 | CATSRDPLNTGELFF |
| 86 | | beta | Variable | MGPGLLHWMALCLLGTGHGDAMVIQN |
| | | | | PRYQVTQFGKPVTLSCSQTLNHNVMYW |
| | | | | YQQKSSQAPKLLFHYYDKDFNNEADTP |
| | | | | DNFQSRRPNTSFCFLDIRSPGLGDAAMY |
| | | | | LCATSRDPLNTGELFFGEGSRLTVL |
| 87 | 1377b | beta | CDR1 | MNHEY |
| | TRBV27*01 | | | |
| | F TRBJ2-1* | | | |
| | 01 F | | | |
| | TRBD2*01 F | | | |
| 88 | | beta | CDR2 | SMNVEV |
| 89 | | beta | CDR3 | CASSSTNYNEQFF |
| 90 | | beta | Variable | MGPQLLGYVVLCLLGAGPLEAQVTQNP |
| | | | | RYLITVTGKKLTVTCSQNMNHEYMSWY |
| | | | | RQDPGLGLRQIYYSMNVEVTDKGDVPE |
| | | | | GYKVSRKEKRNFPLILESPSPNQTSLYFC |
| | | | | ASSSTNYNEQFFGPGTRLTVL |
| 91 | 1378a | alpha | CDR1 | DSAIYN |
| | TRAV21*02 | | | |
| | F TRBJ28*01 | | | |
| | F | | | |
| 92 | | alpha | CDR2 | IQSSQRE |
| 93 | | alpha | CDR3 | CAGAYSGAGSYQLTF |
| 94 | | alpha | Variable | METLLGLLILWLQLQWVSSKQEVTQIPA |
| | | | | ALSVPEGENLVLNCSFTDSAIYNLQWFR |
| | | | | QDPGKGLTSLLLIQSSQREQTSGRLNASL |
| | | | | DKSSGRSTLYIAASQPGDSATYLCAGAY |
| | | | | SGAGSYQLTFGKGTKLSVIPN |
| 95 | 1378a | alpha | CDR1 | DSSSTY |
| | TRAV5*01 F | | | |
| | TRBJ22*01 F | | | |
| 96 | | alpha | CDR2 | IFSNMDM |
| 97 | | alpha | CDR3 | CAEISSGSARQLTF |
| 98 | | alpha | Variable | MKTFAGFSFLFLWLQLDCMSRGEDVEQ |
| | | | | SLFLSVREGDSSVINCTYTDSSSTYLYWY |
| | | | | KQEPGAGLQLLTYIFSNMDMKQDQRLT |
| | | | | VLLNKKDKHLSLRIADTQTGDSAIYFCA |
| | | | | EISSGSARQLTFGSGTQLTVLPD |
| 99 | 1378a | alpha | CDR1 | TSESDYY |
| | TRAV38-2/ | | | |
| | DV8*01 F | | | |
| | TRBJ56*01 F | | | |
| 100 | | alpha | CDR2 | QEAYKQQN |
| 101 | | alpha | CDR3 | CACTGANSKLTF |
| 102 | | alpha | Variable | MACPGFLWALVISTCLEFSMAQTVTQSQ |
| | | | | PEMSVQEAETVTLSCTYDTSESDYYLFW |
| | | | | YKQPPSRQMILVIRQEAYKQQNATENRF |
| | | | | SVNFQKAAKSFSLKISDSQLGDAAMYFC |
| | | | | ACTGANSKLTFGKGITLSVRPD |
| 103 | 1378b | beta | CDR1 | DFQATT |
| | TRBV20-1* | | | |
| | 01 F | | | |
| | TRBJ2-3*01 | | | |
| | F | | | |
| 104 | | beta | CDR2 | SNEGSKA |
| 105 | | beta | CDR3 | CGLAG |
| 106 | | beta | Variable | MLLLLLLLGPGSGLGAVVSQHPSWVICK |
| | | | | SGTSVKIECRSLDFQATTMFWYRQFPKQ |
| | | | | SLMLMATSNEGSKATYEQGVEKDKFLI |
| | | | | NHASLTLSTLTVTSAHPEDSSFYICGLAG |
| 107 | 1375a | alpha | CDR1 | SSVPPY |
| | TRAV8-4*03 | | | |
| | F TRBJ52*01 | | | |
| | F | | | |
| 108 | | alpha | CDR2 | YTTGATLV |
| 109 | | alpha | CDR3 | CAVSDNAGGTSYGKLTF |
| 110 | | alpha | Variable | MLLLLVPVLEVIFTLGGTRAQSVTQLGS |
| | | | | HVSVSEGALVLLRCNYSSSVPPYLFWYV |
| | | | | QYPNQGLQLLLKYTTGATLVKGINGFEA |
| | | | | EFKKSETSFHLTKPSAHMSDAAEYFCAV |
| | | | | SDNAGGTSYGKLTFGQGTILTVHPN |
| 111 | 1375a | alpha | CDR1 | SSYSPS |
| | TRAV8-2*01 | | | |
| | F TRBJ31*01 | | | |
| | F | | | |
| 112 | | alpha | CDR2 | YTSAATLV |
| 113 | | alpha | CDR3 | CVVSDPRDNNARLMF |
| 114 | | alpha | Variable | MLLLLVPVLEVIFTLGGTRAQSVTQLDS |
| | | | | HVSVSEGTPVLLRCNYSSSYSPSLFWYV |
| | | | | QHPNKGLQLLLKYTSAATLVKGINGFEA |
| | | | | EFKKSETSFHLTKPSAHMSDAAEYFCVV |
| | | | | SDPRDNNARLMFGDGTQLVVKPN |
| 115 | 1375a | alpha | CDR1 | YGATPY |
| | TRAV8-3*02 | | | |
| | F TRBJ41*01 | | | |
| | F | | | |
| 116 | | alpha | CDR2 | YFSGDTLV |
| 117 | | alpha | CDR3 | CAVGPNSGYALNF |
| 118 | | alpha | Variable | MLLELIPLLGIHFVLRTARAQSVTQPDIHI |
| | | | | TVSEGASLELRCNYSYGATPYLFWYVQS |
| | | | | PGQGLQLLLKYFSGDTLVQGIKGFEAEF |
| | | | | KRSQSSFNLRKPSVHWSDAAEYFCAVGP |
| | | | | NSGYALNFGKGTSLLVTPH |
| 119 | 1375a | alpha | CDR1 | DSVNN |
| | TRAV22*01 | | | |
| | F TRBJ43*01 | | | |
| | F | | | |
| 120 | | alpha | CDR2 | IPSGT |
| 121 | | alpha | CDR3 | CAVPYNNNDMRF |
| 122 | | alpha | Variable | MKRILGALLGLLSAQVCCVRGIQVEQSP |
| | | | | PDLILQEGANSTLRCNFSDSVNNLQWFH |
| | | | | QNPWGQLINLFYIPSGTKQNGRLSATTV |
| | | | | ATERYSLLYISSSQTTDSGVYFCAVPYN |
| | | | | NNDMRFGAGTRLTVKPN |
| 123 | 1375a | alpha | CDR1 | SSVPPY |
| | TRAV8-4*01 | | | |
| | F TRBJ53*01 | | | |
| | F | | | |
| 124 | | alpha | CDR2 | YTTGATLV |
| 125 | | alpha | CDR3 | CAVSENSGGSNYKLTF |
| 126 | | alpha | Variable | MLLLLVPVLEVIFTLGGTRAQSVTQLGS |
| | | | | HVSVSEGALVLLRCNYSSSVPPYLFWYV |
| | | | | QYPNQGLQLLLKYTTGATLVKGINGFEA |
| | | | | EFKKSETSFHLTKPSAHMSDAAEYFCAV |
| | | | | SENSGGSNYKLTFGKGTLLTVNPN |
| 127 | 1375b | beta | CDR1 | MNHEY |
| | TRBV27-1* | | | |
| | 01 F | | | |
| | TRBJ2-2*01 | | | |
| | F TRBD2*01 | | | |
| | F | | | |
| 128 | | beta | CDR2 | SMNVEV |
| 129 | | beta | CDR3 | CASSPGLNTGELFF |
| 130 | | beta | Variable | MGPQLLGYVVLCLLGAGPLEAQVTQNP |
| | | | | RYLITVTGKKLTVTCSQNMNHEYMSWY |
| | | | | RQDPGLGLRQIYYSMNVEVTDKGDVPE |
| | | | | GYKVSRKEKRNFPLILESPSPNQTSLYFC |
| | | | | ASSPGLNTGELFFGEGSRLTVL |
| 131 | 1375b | beta | CDR1 | MGHAR |
| | TRBV4-1*01 | | | |
| | F TRBJ2-7* | | | |
| | 01 F | | | |
| | TRBD2*01 F | | | |
| 132 | | beta | CDR2 | YSYEKL |
| 133 | | beta | CDR3 | CASSQGTSGFYEQYF |
| 134 | | beta | Variable | MGCRLLCCAVLCLLGAVPIDTEVTQTPK |
| | | | | HLVMGMTNKKSLKCEQHMGHRAMYW |
| | | | | YKQKAKKPPELMFVYSYEKLSINESVPS |
| | | | | RFSPECPNSSLLNLHLHALQPEDSALYLC |
| | | | | ASSQGTSGFYEQYFGPGTRLTVT |
| 135 | 1375b | beta | CDR1 | MNHEY |
| | TRBV27-1* | | | |
| | 01 F | | | |
| | TRBJ2-1*01 | | | |
| | F | | | |
| 136 | | beta | CDR2 | SMNVEV |
| 137 | | beta | CDR3 | CASSLSNYNEQFF |
| 138 | | beta | Variable | MGPQLLGYVVLCLLGAGPLEAQVTQNP |
| | | | | RYLITVTGKKLTVTCSQNMNHEYMSWY |
| | | | | RQDPGLGLRQIYYSMNVEVTDKGDVPE |
| | | | | GYKVSRKEKRNFPLILESPSPNQTSLYFC |
| | | | | ASSLSNYNEQFFGPGTRLTVL |
| 139 | 3748a | alpha | CDR1 | NIATNDY |
| | TRAV4*01 F | | | |
| | TRBJ39*01 F | | | |
| 140 | | alpha | CDR2 | GYKTK |
| 141 | | alpha | CDR3 | CLVGAGGNNAGNMLTF |
| 142 | | alpha | Variable | MRQVARVIVFLTLSTLSLAKTTQPISMDS |
| | | | | YEGQEVNITCSHNNIATNDYITWYQQFP |
| | | | | SQGPRFIIQGYKTKVTNEVASLFIPADRK |
| | | | | SSTLSLPRVSLSDTAVYYCLVGAGGNNA |
| | | | | GNMLTFGGGTRLMVKPH |
| 143 | 3748a | alpha | CDR1 | DSASNY |
| | TRAV13-1* | | | |
| | 01 F | | | |
| | TRBJ50* | | | |
| | 01 F | | | |
| 144 | | alpha | CDR2 | IRSNVGE |
| 145 | | alpha | CDR3 | CAASMKTSYDKVIF |
| 146 | | alpha | Variable | MTSIRAVFIFLWLQLDLVNGENVEQHPS |
| | | | | TLSVQEGDSAVIKCTYSDSASNYFPWYK |
| | | | | QELGKGPQLIIDIRSNVGEKKDQRIAVTL |
| | | | | NKTAKHFSLHITETQPEDSAVYFCAASM |
| | | | | KTSYDKVIFGPGTSLSVIPN |
| 147 | 3748a | alpha | CDR1 | SSNFYA |
| | TRAV24*01 | | | |
| | F TRBJ34*01 | | | |
| | F | | | |
| 148 | | alpha | CDR2 | MTLNGDE |
| 149 | | alpha | CDR3 | CAPIYNTDKLIF |
| 150 | | alpha | Variable | MEKNPLAAPLLILWFHLDCVSSILNVEQ |
| | | | | SPQSLHVQEGDSTNFTCSFPSSNFYALH |
| | | | | WYRWETAKSPEALFVMTLNGDEKKKG |
| | | | | RISATLNTKEGYSYLYIKGSQPEDSATYL |
| | | | | CAPIYNTDKLIFGTGTRLQVFPN |
| 151 | 3748a | alpha | CDR1 | TSESNYY |
| | TRAV38-1* | | | |
| | 03 F | | | |
| | TRBJ56* | | | |
| | 01 F | | | |
| 152 | | alpha | CDR2 | QEAYKQQN |
| 153 | | alpha | CDR3 | CAFMTDTGANSKLTF |
| 154 | | alpha | Variable | MGTLQGSAVSMTRVSLLWAVVVSTCLE |
| | | | | SGMAQTVTQSQPEMSVQEAETVTLSCT |
| | | | | YDTSESNYYLFWYKQPPSRQMILVIRQE |
| | | | | AYKQQNATENRFSVNFQKAAKSFSLKIS |
| | | | | DSQLGDTAMYFCAFMTDTGANSKLTFG |
| | | | | KGITLSVRPD |
| 155 | 3748b | beta | CDR1 | SGHNS |
| | TRBV12-3* | | | |
| | 01 F | | | |
| | TRBJ2-7*01 | | | |
| | F TRBD2*01 | | | |
| | F | | | |
| 156 | | beta | CDR2 | FNNNVP |
| 157 | | beta | CDR3 | CASSLGGGSYEQYF |
| 158 | | beta | Variable | MDSWTFCCVSLCILVAKHTDAGVIQSPR |
| | | | | HEVTEMGQEVTLRCKPISGHNSLFWYR |
| | | | | QTMMRGLELLIYFNNNVPIDDSGMPEDR |
| | | | | FSAKMPNASFSTLKIQPSEPRDSAVYFCA |
| | | | | SSLGGGSYEQYFGPGTRLTVT |
| 159 | 3748b | beta | CDR1 | KAHSY |
| | TRBV21- | | | |
| | 1*01 F | | | |
| | TRBJ2-2*01 | | | |
| | F TRBD1*01 | | | |
| | F | | | |
| 160 | | beta | CDR2 | FQNEEL |
| 161 | | beta | CDR3 | CASSKTGTANTGELFF |
| 162 | | beta | Variable | MDCVPIKAHSYVYWYRKKLEEELKFLV |
| | | | | YFQNEELIQKAEIINERFLAQCSKNSSCT |
| | | | | LEIQSTESGDTALYFCASSKTGTANTGEL |
| | | | | FFGEGSRLTVL |

TABLE 2
Peptide sequences of the invention

| Peptide Code | Sequence | SEQ ID NO: |
|---|---|---|
| Kras minimal epitope | KLVVVGAVGV | 73 |
| Kras C-terminal extension | KLVVVGAVGVG | 74 |
| Kras N-terminal extension | TEYKLVVVGAVGV | 75 |
| Kras spliced epitope 1 | KLVVGAVGV | 76 |
| Kras spliced epitope 2 | KLVVVAVGV | 77 |
| Kras spliced epitope 3 | YLVVVGAVGV | 78 |
| Kras spliced epitope 4 | KLVVVGVGV | 163 |

### EXAMPLES

### Example 1

### Mutant RAS Specific TCRs for Immunotherapy

The spliced epitopes carrying the Ras^{G12V} mutation were identified by combining in silico predictions to in vitro experiments measured by mass spectrometry. Briefly, the inventors developed an algorithm able to predict all possible spliced peptides derived from a given sequence, carrying a given residue (in this case, the residue V in position 12 of human KRAS). The algorithm then ranked the spliced peptide candidates by predicting their binding affinity to the target HLA-I allele (in this case, HLA-A*02:01). Afterwards, the algorithm predicts the binding affinity to the same HLA-I allele for the spliced peptide candidates in which the mutated target residue has been exchanged with the wild type residue (in this case, the G12 in human KRAS). In case the wild type spliced peptide had a predicted similar (or better) affinity to the HLA-I allele, the corresponding mutated spliced peptide candidate is eliminated from the list. By developing and applying this algorithm the inventors generated a list of spliced epitope candidates carrying the RAS G12V mutation (see below), efficiently binding the HLA-A*02:01 molecule and having a wild type spliced peptide counterpart that is supposed to bind less efficiently the HLA-A*02:01 molecule. From this list the inventors took the best 10 candidates, prepared a m/z inclusion list and used it to streamline the mass spectrometry (MS) analysis of in vitro digestion of the synthetic precursor substrate Ras^{G12V} (aa 2-35) by purified 20S proteasome. A variant of the algorithm used for this quest has been developed and applied to identify spliced epitopes triggering an immune response during Listeria monocytogenes infection in a mouse model (Platteel et al., Cell Rep. 2017). The in vitro digestions was carried out as described elsewhere (Liepe et al., Science 2016).

MS data were collected using an Orbitrap Fusion Lumos mass spectrometer coupled to an Ultimate 3000 RSLC nano pump (both from ThermoFisherScientific).

In a brief, peptides were loaded and separated by a nanoflow HPLC (RSLC Ultimate 3000) on an Easy-spray C18 nano column (so cm length, 75 mm internal diameter; ThermoFisherScientific) coupled on-line to a nano-electrospray ionization Orbitrap Fusion Lumos mass spectrometer (ThermoFisherScientific). Peptides were eluted with a linear gradient of 2%-45% buffer B (80% ACN, 0.05% formic acid) at a flow rate of 250 nl/min over 60 min at 50° C.

The instrument was programmed within Xcalibur 4.1 to acquire MS data using a “Universal” method by defining a 3s cycle time between a full MS scan and MS/MS fragmentation. This method takes advantage of multiple analyzers in the Orbitrap Fusion Lumos and drives the system to use all available parallelizable time, resulting in decreasing the dependence on method parameters (such as Data Dependent Acquisition; DDA). The inventors acquired one full-scan MS spectrum at a resolution of 120,000 at 200 m/z with an automatic gain control (AGC) target value of 2xe5 ions and a scan range of 350˜1550 m/z. The MS/MS fragmentation was conducted using HCD collision energy (30%) with an orbitrap resolution of 30000 at Zoom/z. The AGC target value was set up as 5xe4 with a max injection time of 120 ms. A dynamic exclusion of 30 s and 1-4 included charged states were defined within this method.

By MS the inventors identified the SEQ. 76-78 as well as some of the N-terminal and C-terminal elongated spliced peptides, which were included in the inclusion list.

Alternative mass spectrometry methods have been applied to confirm the correct identification of the peptides.

As last step the inventors applied an algorithm aimed to exclude from the final list of the identified spliced peptides all artefacts, which are very frequent in this type of pipeline. This last step of filtering out all artefacts is based on kinetics biochemical experiments.

The following sequences were identified:

minimal epitope: KLVVVGAVGV (10 mer, linear)
C-terminal extension: KLVVVGAVGVG (11 mer)
N-terminal extension: TEYKLVVVGAVGV (13 mer)
spliced epitope 1: KLVVGAVGV (S-6_8-14) (9 mer)
spliced epitope 2: KLVVVAVGV (S-9_11-14) (9 mer)
spliced epitope 3: YLVVVGAVGV (4_6-14) (10 mer)
and
further:
spliced epitope 4: KLVVVGVGV (9 mer)

Spliced epitope 4 was generated by:
In silico analysis of KRAS2-15 KRAS 2-21 G12V protein sequence with ProtAG algorithm and neospliceepitope prediction,
In vitro prozessing of KRAS G12V deduced synthetic polypeptides KRAS 2-15 KRAS 2-21 using purified human 20S proteasome,
Mass spectrometric identification of neosplicetopes using Orbitrap
QExactive

Example of the kinetics of in vitro KLVVGAVGV spliced peptide generated by human 20S standard (triangles) and immunoproteasome (squares) from mutKras G12V delineated synthetic polypeptides mutKras 2-21 or mutKras 2-14 is shown in FIG. 7A. Respective synthetic polypeptides were incubated with purified 20S proteasomes derived from human T2 cells (standard proteasome) and T27 cells (immuno-proteasomes) for different time points. SpiG12V was identified by mass spectrometry using an Orbitrap XL or QE Exactive supported by a spliced peptide inclusion list generated by the in house ProtAG algorithm in combination with NetMHCpan 4.0 permitting the prediction of theoretically generated immune relevant mutKras G12V derived spliced neoepitopes. The kinetic experiment reveals the efficient in vitro generation of KLVVGAVGV by both proteasome subtypes.

Comparison of the MSMS spectra of the KLVVGAVGV (K5 L6 V7 V8 G10 Au V12 G13 V14) generated in vitro by 20S proteasomes (A) and of the corresponding synthetic polypeptide KLVVGAVGV. The MSMS spectrum of the KLVVGAVGV synthetic peptide confirms the correctness of the amino acid sequence of KLVVGAVGV spliced epitope generated by 20S proteasomes and identified in in vitro digests (FIGS. 7B and C).

The generation kinetics of KLVVVAVGV by standard (red) and immunoproteasomes (blue) was analysed and monitored as described in FIG. 7A. E: The amino acid sequence of the by ProtAG/NetMHCpan4.0 predicted KrasG12V derived spliced peptide generated in vitro by both 20S proteasome subtypes was confirmed by MSMS (FIG. 7D and E).

The generation kinetics of KLVVVGVGV (FIG. 7F) by standard (triangle) and immunoproteasomes (squares) was analysed and monitored as described in FIG. 7A. The amino acid sequence of the by ProtAG/NetMHCpan4.0 predicted KrasG12V derived spliced peptide generated in vitro by both 20S proteasome subtypes was confirmed by MSMS (FIG. 7G).

TABLE 3
Binding affinity of spliced
epitopes to HLA-A*02:01.

| | IC50 | Designation: |
|---|---|---|
| G12wt | | |
| KLVVVGAGGV | 405 | lin wt |
| G12V | | |
| KLVVVGAVGV | 157 | lin |
| KLVVGAVGV | 33 | spi |
| KLVVVAVGV | 38 | 5p2 |
| YLVVVGAVGV | 53 | 5p3 |
| KLVVVGVGV | 73 | 5p4 |

In comparison to the linear wildtype an mutant epitope of Kras in silico analysis of spliced epitopes using NetMHCpan 4.0 prediction software assumes higher affinity (IC₅₀) of the spliced epitopes to bind to HLA-A*02:01.

### Example 2

For one Ras^{G12V} specific TCR recognizing spliced peptide KLVVGAVGV we could show reactivity to very low peptide concentrations (FIG. 2) and recognition of endogenously processed and presented epitope in human colon cell lines (FIG. 4). The respective Ras^{G12V} specific TCR does not recognize high concentrations (10⁻⁶ and 10⁻⁸ M) of Ras peptides (linear and spliced, FIGS. 3 and 5) but cross-reacts to mutant linear Ras^{G12V} peptide KLVVVGAYGV and spliced peptides carrying G12S/A/C substitutions (FIG. 5). Supposedly due to central tolerance the spliced Ras^{wt} epitope KLVVVGAGGV is not immunogenic in ABabDII mice (FIG. 6)

1-28. (canceled)

29. An antigen recognizing construct, comprising at least one complementary determining region which specifically recognizes a mutated Ras antigen such as a mutated Ras^{G12}, preferably wherein the spliced peptide variant comprises a sequence according to SEQ ID NO: 76 to 78, or 163.

30. The antigen recognizing construct according to claim 29, comprising at least one complementary determining region (CDR) 3 having at least 80% sequence identity to an amino acid sequence selected from SEQ ID NOs. 3, 7, 11, 15, 19, 23, 27, 31, 35, 39, 43, 47, 51, 55, 59, 63, 67, and 71, or a CDR3 shown in table 1a in each case independently, optionally with not more than three or two, preferably no more than one, amino acid substitution(s), insertion(s) or deletion(s) compared to these sequences.

31. The antigen recognizing construct according to claim 29, wherein the antigen recognizing construct is an α/β-TCR, or fragment or derivative thereof, or the construct is a γ/δ-TCR, or a fragment or derivative thereof.

32. The antigen recognizing construct according to claim 29, comprising a TCR α or γ chain; and/or a TCR β or δ chain; wherein the TCR α or γ chain comprises a CDR3 having at least 80% sequence identity to an amino acid sequence selected from SEQ ID Nos. 3, 11, 19, 27, 35, 43, 51, 59, and 67 or an alpha chain CDR3 shown in table 1a, and/or wherein the TCR β or δ chain comprises a CDR3 having at least 80% sequence identity to an amino acid sequence selected from SEQ ID Nos. 7, 15, 23, 31, 39, 47, 55, 63, and 71 or a beta chain CDR3 shown in table 1a; in each case independently, optionally with not more than three or two, preferably no more than one, amino acid substitution(s), insertion(s) or deletion(s) compared to these sequences.

33. The antigen recognizing construct according to claim 29, comprising a TCR variable chain region having at least 80% sequence identity to an amino acid sequence selected from SEQ ID Nos. 4, 8, 12, 16, 20, 24, 28, 32, 36, 40, 44, 48, 52, 56, 60, 64, 68, and 72 or a variable domain sequence shown in table 1a, in each case independently, optionally with not more than three or two, preferably no more than one, amino acid substitution(s), insertion(s) or deletion(s) compared to these sequences.

34. The antigen recognizing construct according to claim 29, wherein the construct is fully or partially humanized, chimerized and/or murinized.

35. The antigen recognizing construct according to claim 29, wherein the construct is a TCR, or a fragment thereof, composed of at least one TCR α and one TCR β chain sequence, wherein said TCR α chain sequence and said TCR β chain sequence is selected from the following combinations:

| Alpha Chain CDR1, CDR2, CDR3 | | | Beta Chain CDR1, CDR2, CDR3 | | |
|---|---|---|---|---|---|
| (SEQ ID NO) | | | (SEQ ID NO) | | |
| 1 | 2 | 3 | 5 | 6 | 7 |
| 9 | 10 | 11 | 13 | 14 | 15 |
| 17 | 18 | 19 | 21 | 22 | 23 |
| 25 | 26 | 27 | 29 | 30 | 31 |
| 33 | 34 | 35 | 37 | 38 | 39 |
| 41 | 42 | 43 | 45 | 46 | 47 |
| 49 | 50 | 51 | 53 | 54 | 55 |
| 57 | 58 | 59 | 61 | 62 | 63 |
| 65 | 66 | 67 | 69 | 70 | 71 |
| 1 | 2 | 3 | 79 | 80 | 81 |
| 9 | 10 | 11 | 83 | 84 | 85 |
| 9 | 10 | 11 | 87 | 88 | 89 |
| 91 | 92 | 93 | 103 | 104 | 105 |
| 95 | 96 | 97 | 103 | 104 | 105 |
| 99 | 100 | 101 | 103 | 104 | 105 |
| 17 | 18 | 19 | 103 | 104 | 105 |
| 91 | 92 | 93 | 21 | 22 | 23 |
| 95 | 96 | 97 | 21 | 22 | 23 |
| 99 | 100 | 101 | 21 | 22 | 23 |
| 107 | 108 | 109 | 127 | 128 | 129 |
| 107 | 108 | 109 | 131 | 132 | 133 |
| 107 | 108 | 109 | 135 | 136 | 137 |
| 111 | 112 | 113 | 127 | 128 | 129 |
| 111 | 112 | 113 | 131 | 132 | 133 |
| 111 | 112 | 113 | 135 | 136 | 137 |
| 115 | 116 | 117 | 127 | 128 | 129 |
| 115 | 116 | 117 | 131 | 132 | 133 |
| 115 | 116 | 117 | 135 | 136 | 137 |
| 119 | 120 | 121 | 127 | 128 | 129 |
| 119 | 120 | 121 | 131 | 132 | 133 |
| 119 | 120 | 121 | 135 | 136 | 137 |
| 123 | 124 | 125 | 127 | 128 | 129 |
| 123 | 124 | 125 | 131 | 132 | 133 |
| 123 | 124 | 125 | 135 | 136 | 137 |
| 139 | 140 | 141 | 155 | 156 | 157 |
| 139 | 140 | 141 | 159 | 160 | 161 |
| 143 | 144 | 145 | 155 | 156 | 157 |
| 143 | 144 | 145 | 159 | 160 | 161 |
| 147 | 148 | 149 | 155 | 156 | 157 |
| 147 | 148 | 149 | 159 | 160 | 161 |
| 151 | 152 | 153 | 155 | 156 | 157 |
| 151 | 152 | 153 | 159 | 160 | 161 |

in each case independently, optionally with not more than three or two, preferably no more than one, amino acid substitution(s), insertion(s) or deletion(s) compared to these sequences.

36. The antigen recognizing construct according to claim 29, wherein the construct is a TCR, or a fragment thereof, further comprising a TCR constant region preferably a human or mouse TCR constant region.

37. A nucleic acid encoding for an antigen recognizing construct comprising at least one complementary determining region which specifically recognizes a mutated Ras antigen derived spliced peptide variant which comprises a sequence according to SEQ ID NO: 76 to 78, or 163.

38. A vector comprising a nucleic acid according to claim 37.

39. A host cell comprising an antigen recognizing construct according to claim 29.

40. A pharmaceutical composition comprising the antigen recognizing construct according to claim 29, and a pharmaceutical acceptable carrier, stabilizer and/or excipient.

41. A method for the treatment of a proliferative disease in a subject, comprising a step of administering to the subject an antigen recognizing construct, wherein the disease comprises a malignant or benign tumor disease, preferably a tumor disease which is positive for the Ras^{G12V} mutation, and wherein the antigen recognizing construct comprising at least one complementary determining region which specifically recognizes a mutated Ras antigen derived spliced peptide variant which comprises a sequence according to SEQ ID NO: 76 to 78, or 163.

42. The method of claim 41, wherein the treatment is an immune therapy, optionally, comprising an adoptive cell transfer, wherein the immune therapy comprises adoptive autologous or heterologous T-cell therapy.

43. A method of manufacturing a TSA specific antigen recognizing construct expressing cell line, comprising
a. providing a suitable host cell,
b. providing a genetic construct comprising a coding sequence encoding the antigen recognizing construct according to of claim 29,
c. introducing into said suitable host cell said genetic construct, and
d. expressing said genetic construct by said suitable host cell.

44. An immunogenic peptide that is selected from the group of peptides comprising at least on sequence according to any of SEQ ID No. 76 to 78, or 163, or a variant thereof.

45. The immunogenic peptide according to claim 44, wherein the peptide consists essentially of an amino acid sequence according to any of SEQ ID No. 76 to 78, or 163, or a variant thereof.

46. The immunogenic peptide according to claim 44, wherein said peptide exhibits an overall length of between 9 and 100, preferably between 9 and 30 amino acids.

47. The immunogenic peptide according to claim 44, consisting of an amino acid sequence according to any of SEQ ID No. 76 to 78, or SEQ ID No. 163.

48. The immunogenic peptide according to claim 44, having the ability to bind to a molecule of the human major histocompatibility complex (MHC) class-I, in particular to HLA-A*02.

49. A nucleic acid, encoding a peptide selected from the group of peptides comprising at least on sequence according to any of SEQ ID No. 76 to 78, or 163, or a variant thereof.

50. A host cell comprising a nucleic acid according to claim 49.

51. A pharmaceutical composition comprising an immunogenic peptide selected from the group of peptides comprising at least on sequence according to any of SEQ ID No. 76 to 78, or 163, or a variant thereof, and a pharmaceutically acceptable carrier.

52. A T-cell receptor (TCR) which recognizes a cell which aberrantly expresses a mutated Ras antigen, the TCR being obtainable from the cytotoxic T lymphocyte (CTL) reactive to an immunogenic peptide comprising at least on sequence according to any of SEQ ID No. 76 to 78, or 163, or a variant thereof.


SEQUENCE LISTING (163 sequences)

SEQ ID NO: 1 - protein, 6 aa, Homo sapiens
DSSSTY

SEQ ID NO: 2 - protein, 7 aa, Homo sapiens
IFSNMDM

SEQ ID NO: 3 - protein, 13 aa, Homo sapiens
CAESTDSWGKLQF

SEQ ID NO: 4 - protein, 133 aa, Homo sapiens
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAESTDSWGKLQFGAGTQVVVTPD

SEQ ID NO: 5 - protein, 5 aa, Homo sapiens
MGHRA

SEQ ID NO: 6 - protein, 6 aa, Homo sapiens
YSYEKL

SEQ ID NO: 7 - protein, 14 aa, Homo sapiens
CASSQDLAGYEQYF

SEQ ID NO: 8 - protein, 132 aa, Homo sapiens
MGCRLLCCAVLCLLGAVPIDTEVTQTPKHLVMGMTNKKSLKCEQHMGHRAMYWYKQKAKKPPELMFVYSYEKLSINESVPSRFSPECPNSSLLNLHLHALQPEDSALYLCASSQDLAGYEQYFGPGTRLTVT

SEQ ID NO: 9 - protein, 6 aa, Homo sapiens
NSAFQY

SEQ ID NO: 10 - protein, 6 aa, Homo sapiens
TYSSGN

SEQ ID NO: 11 - protein, 11 aa, Homo sapiens
CAIFSGSRLTF

SEQ ID NO: 12 - protein, 132 aa, Homo sapiens
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAIFSGSRLTFGEGTQLTVNPD

SEQ ID NO: 13 - protein, 5 aa, Homo sapiens
SQVTM

SEQ ID NO: 14 - protein, 7 aa, Homo sapiens
ANQGSEA

SEQ ID NO: 15 - protein, 16 aa, Homo sapiens
CSVAGLAGSSYNEQFF

SEQ ID NO: 16 - protein, 132 aa, Homo sapiens
MLSLLLLLLGLGSVFSAVISQKPSRDICQRGTSLTIQCQVDSQVTMMFWYRQQPGQSLTLIATANQGSEATYESGFVIDKFPISRPNLTFSTLTVSNMSPEDSSIYLCSVAGLAGSSYNEQFFGPGTRLTVL

SEQ ID NO: 17 - protein, 5 aa, Homo sapiens
TTSDR

SEQ ID NO: 18 - protein, 7 aa, Homo sapiens
LLSNGAV

SEQ ID NO: 19 - protein, 12 aa, Homo sapiens
CAGIQGAQKLVF

SEQ ID NO: 20 - protein, 129 aa, Homo sapiens
MKKLLAMILWLQLDRLSGELKVEQNPLFLSMQEGKNYTIYCNYSTTSDRLYWYRQDPGKSLESLFVLLSNGAVKQEGRLMASLDTKARLSTLHITAAVHDLSATYFCAGIQGAQKLVFGQGTRLTINPN

SEQ ID NO: 21 - protein, 5 aa, Homo sapiens
MNHEY

SEQ ID NO: 22 - protein, 6 aa, Homo sapiens
SMNVEV

SEQ ID NO: 23 - protein, 13 aa, Homo sapiens
CASSLWTNNEQFF

SEQ ID NO: 24 - protein, 131 aa, Homo sapiens
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSLWTNNEQFFGPGTRLTVL

SEQ ID NO: 25 - protein, 5 aa, Homo sapiens
TSINN

SEQ ID NO: 26 - protein, 7 aa, Homo sapiens
IRSNERE

SEQ ID NO: 27 - protein, 13 aa, Homo sapiens
CATDEDTGNQFYF

SEQ ID NO: 28 - protein, 132 aa, Homo sapiens
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDEDTGNQFYFGTGTSLTVIPN

SEQ ID NO: 29 - protein, 5 aa, Homo sapiens
SGHNS

SEQ ID NO: 30 - protein, 6 aa, Homo sapiens
FNNNVP

SEQ ID NO: 31 - protein, 12 aa, Homo sapiens
CASSLWGYEQYF

SEQ ID NO: 32 - protein, 131 aa, Homo sapiens
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSLWGYEQYFGPGTRLTVT

SEQ ID NO: 33 - protein, 5 aa, Homo sapiens
TSINN

SEQ ID NO: 34 - protein, 7 aa, Homo sapiens
IRSNERE

SEQ ID NO: 35 - protein, 13 aa, Homo sapiens
CATDEDTGNQFYF

SEQ ID NO: 36 - protein, 132 aa, Homo sapiens; identical to SEQ ID NO: 28

SEQ ID NO: 37 - protein, 5 aa, Homo sapiens
SGHNS

SEQ ID NO: 38 - protein, 6 aa, Homo sapiens
FNNNVP

SEQ ID NO: 39 - protein, 12 aa, Homo sapiens
CASSLVGYEQYF

SEQ ID NO: 40 - protein, 131 aa, Homo sapiens
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSLVGYEQYFGPGTRLTVT

SEQ ID NO: 41 - protein, 7 aa, Homo sapiens
TSESDYY

SEQ ID NO: 42 - protein, 8 aa, Homo sapiens
QEAYKQQN

SEQ ID NO: 43 - protein, 12 aa, Homo sapiens
CAFGGSNYKLTF

SEQ ID NO: 44 - protein, 134 aa, Homo sapiens
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAFGGSNYKLTFGKGTLLTVNPN

SEQ ID NO: 45 - protein, 5 aa, Homo sapiens
LNHNV

SEQ ID NO: 46 - protein, 6 aa, Homo sapiens
YYDKDF

SEQ ID NO: 47 - protein, 12 aa, Homo sapiens
CATSGSQNIQYF

SEQ ID NO: 48 - protein, 130 aa, Homo sapiens
MGPGLLHWMALCLLGTGHGDAMVIQNPRYQVTQFGKPVTLSCSQTLNHNVMYWYQQKSSQAPKLLFHYYDKDFNNEADTPDNFQSRRPNTSFCFLDIRSPGLGDAAMYLCATSGSQNIQYFGAGTRLSVL

SEQ ID NO: 49 - protein, 7 aa, Homo sapiens
TSESDYY

SEQ ID NO: 50 - protein, 8 aa, Homo sapiens
QEAYKQQN

SEQ ID NO: 51 - protein, 12 aa, Homo sapiens
CAFGGSNYKLTF

SEQ ID NO: 52 - protein, 134 aa, Homo sapiens; identical to SEQ ID NO: 44

SEQ ID NO: 53 - protein, 5 aa, Homo sapiens
MDHEN

SEQ ID NO: 54 - protein, 6 aa, Homo sapiens
SYDVKM

SEQ ID NO: 55 - protein, 13 aa, Homo sapiens
CASSQGLALEQFF

SEQ ID NO: 56 - protein, 131 aa, Homo sapiens
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSQGLALEQFFGPGTRLTVL

SEQ ID NO: 57 - protein, 6 aa, Homo sapiens
SSVSVY

SEQ ID NO: 58 - protein, 8 aa, Homo sapiens
YLSGSTLV

SEQ ID NO: 59 - protein, 13 aa, Homo sapiens
CAVSGGSNYKLTF

SEQ ID NO: 60 - protein, 133 aa, Homo sapiens
MLLLLVPAFQVIFTLGGTRAQSVTQLDSQVPVFEEAPVELRCNYSSSVSVYLFWYVQYPNQGLQLLLKYLSGSTLVESINGFEAEFNKSQTSFHLRKPSVHISDTAEYFCAVSGGSNYKLTFGKGTLLTVNPN

SEQ ID NO: 61 - protein, 5 aa, Homo sapiens
LGHNA

SEQ ID NO: 62 - protein, 6 aa, Homo sapiens
YNFKEQ

SEQ ID NO: 63 - protein, 16 aa, Homo sapiens
CASSQESGNLYNEQFF

SEQ ID NO: 64 - protein, 134 aa, Homo sapiens
MGCRLLCCAVLCLLGAVPMETGVTQTPRHLVMGMTNKKSLKCEQHLGHNAMYWYKQSAKKPLELMFVYNFKEQTENNSVPSRFSPECPNSSHLFLHLHTLQPEDSALYLCASSQESGNLYNEQFFGPGTRLTVL

SEQ ID NO: 65 - protein, 6 aa, Homo sapiens
NSMFDY

SEQ ID NO: 66 - protein, 7 aa, Homo sapiens
ISSIKDK

SEQ ID NO: 67 - protein, 14 aa, Homo sapiens
CAASRWDTGNQFYF

SEQ ID NO: 68 - protein, 140 aa, Homo sapiens
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASRWDTGNQFYFGTGTSLTVIPN

SEQ ID NO: 69 - protein, 5 aa, Homo sapiens
MDHEN

SEQ ID NO: 70 - protein, 6 aa, Homo sapiens
SYDVKM

SEQ ID NO: 71 - protein, 14 aa, Homo sapiens
CASSWTSGYNEQFF

SEQ ID NO: 72 - protein, 132 aa, Homo sapiens
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSWTSGYNEQFFGPGTRLTVL

SEQ ID NO: 73 - protein, 10 aa, Homo sapiens
KLVVVGAVGV

SEQ ID NO: 74 - protein, 11 aa, Homo sapiens
KLVVVGAVGVG

SEQ ID NO: 75 - protein, 13 aa, Homo sapiens
TEYKLVVVGAVGV

SEQ ID NO: 76 - protein, 9 aa, Homo sapiens
KLVVGAVGV

SEQ ID NO: 77 - protein, 9 aa, Homo sapiens
KLVVVAVGV

SEQ ID NO: 78 - protein, 10 aa, Homo sapiens
YLVVVGAVGV

SEQ ID NO: 79 - protein, 5 aa, Homo sapiens
LGHRA

SEQ ID NO: 80 - protein, 6 aa, Homo sapiens
YSYEKL

SEQ ID NO: 81 - protein, 14 aa, Homo sapiens
CASSQDLAGYEQYF

SEQ ID NO: 82 - protein, 132 aa, Homo sapiens
MGCRLLCCAVLCLLGAVPIDTEVTQTPKHLVMGMTNKKSLKCEQHLGHRAMYWYKQKAKKPPELMFVYSYEKLSINESVPSRFSPECPNSSLLNLHLHALQPEDSALYLCASSQDLAGYEQYFGPGTRLTVT

SEQ ID NO: 83 - protein, 5 aa, Homo sapiens
LNHNV

SEQ ID NO: 84 - protein, 6 aa, Homo sapiens
YYDKDF

SEQ ID NO: 85 - protein, 15 aa, Homo sapiens
CATSRDPLNTGELFF

SEQ ID NO: 86 - protein, 133 aa, Homo sapiens
MGPGLLHWMALCLLGTGHGDAMVIQNPRYQVTQFGKPVTLSCSQTLNHNVMYWYQQKSSQAPKLLFHYYDKDFNNEADTPDNFQSRRPNTSFCFLDIRSPGLGDAAMYLCATSRDPLNTGELFFGEGSRLTVL

SEQ ID NO: 87 - protein, 5 aa, Homo sapiens
MNHEY

SEQ ID NO: 88 - protein, 6 aa, Homo sapiens
SMNVEV

SEQ ID NO: 89 - protein, 13 aa, Homo sapiens
CASSSTNYNEQFF

SEQ ID NO: 90 - protein, 131 aa, Homo sapiens
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSSTNYNEQFFGPGTRLTVL

SEQ ID NO: 91 - protein, 6 aa, Homo sapiens
DSAIYN

SEQ ID NO: 92 - protein, 7 aa, Homo sapiens
IQSSQRE

SEQ ID NO: 93 - protein, 15 aa, Homo sapiens
CAGAYSGAGSYQLTF

SEQ ID NO: 94 - protein, 134 aa, Homo sapiens
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAGAYSGAGSYQLTFGKGTKLSVIPN

SEQ ID NO: 95 - protein, 6 aa, Homo sapiens
DSSSTY

SEQ ID NO: 96 - protein, 7 aa, Homo sapiens
IFSNMDM

SEQ ID NO: 97 - protein, 14 aa, Homo sapiens
CAEISSGSARQLTF

SEQ ID NO: 98 - protein, 134 aa, Homo sapiens
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAEISSGSARQLTFGSGTQLTVLPD

SEQ ID NO: 99 - protein, 7 aa, Homo sapiens
TSESDYY

SEQ ID NO: 100 - protein, 8 aa, Homo sapiens
QEAYKQQN

SEQ ID NO: 101 - protein, 12 aa, Homo sapiens
CACTGANSKLTF

SEQ ID NO: 102 - protein, 134 aa, Homo sapiens
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCACTGANSKLTFGKGITLSVRPD

SEQ ID NO: 103 - protein, 6 aa, Homo sapiens
DFQATT

SEQ ID NO: 104 - protein, 7 aa, Homo sapiens
SNEGSKA

SEQ ID NO: 105 - protein, 5 aa, Homo sapiens
CGLAG

SEQ ID NO: 106 - protein, 112 aa, Homo sapiens
MLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICGLAG

SEQ ID NO: 107 - protein, 6 aa, Homo sapiens
SSVPPY

SEQ ID NO: 108 - protein, 8 aa, Homo sapiens
YTTGATLV

SEQ ID NO: 109 - protein, 17 aa, Homo sapiens
CAVSDNAGGTSYGKLTF

SEQ ID NO: 110 - protein, 137 aa, Homo sapiens
MLLLLVPVLEVIFTLGGTRAQSVTQLGSHVSVSEGALVLLRCNYSSSVPPYLFWYVQYPNQGLQLLLKYTTGATLVKGINGFEAEFKKSETSFHLTKPSAHMSDAAEYFCAVSDNAGGTSYGKLTFGQGTILTVHPN

SEQ ID NO: 111 - protein, 6 aa, Homo sapiens
SSYSPS

SEQ ID NO: 112 - protein, 8 aa, Homo sapiens
YTSAATLV

SEQ ID NO: 113 - protein, 15 aa, Homo sapiens
CVVSDPRDNNARLMF

SEQ ID NO: 114 - protein, 135 aa, Homo sapiens
MLLLLVPVLEVIFTLGGTRAQSVTQLDSHVSVSEGTPVLLRCNYSSSYSPSLFWYVQHPNKGLQLLLKYTSAATLVKGINGFEAEFKKSETSFHLTKPSAHMSDAAEYFCVVSDPRDNNARLMFGDGTQLVVKPN

SEQ ID NO: 115 - protein, 6 aa, Homo sapiens
YGATPY

SEQ ID NO: 116 - protein, 8 aa, Homo sapiens
YFSGDTLV

SEQ ID NO: 117 - protein, 13 aa, Homo sapiens
CAVGPNSGYALNF

SEQ ID NO: 118 - protein, 133 aa, Homo sapiens
MLLELIPLLGIHFVLRTARAQSVTQPDIHITVSEGASLELRCNYSYGATPYLFWYVQSPGQGLQLLLKYFSGDTLVQGIKGFEAEFKRSQSSFNLRKPSVHWSDAAEYFCAVGPNSGYALNFGKGTSLLVTPH

SEQ ID NO: 119 - protein, 5 aa, Homo sapiens
DSVNN

SEQ ID NO: 120 - protein, 5 aa, Homo sapiens
IPSGT

SEQ ID NO: 121 - protein, 12 aa, Homo sapiens
CAVPYNNNDMRF

SEQ ID NO: 122 - protein, 129 aa, Homo sapiens
MKRILGALLGLLSAQVCCVRGIQVEQSPPDLILQEGANSTLRCNFSDSVNNLQWFHQNPWGQLINLFYIPSGTKQNGRLSATTVATERYSLLYISSSQTTDSGVYFCAVPYNNNDMRFGAGTRLTVKPN

SEQ ID NO: 123 - protein, 6 aa, Homo sapiens
SSVPPY

SEQ ID NO: 124 - protein, 8 aa, Homo sapiens
YTTGATLV

SEQ ID NO: 125 - protein, 16 aa, Homo sapiens
CAVSENSGGSNYKLTF

SEQ ID NO: 126 - protein, 136 aa, Homo sapiens
MLLLLVPVLEVIFTLGGTRAQSVTQLGSHVSVSEGALVLLRCNYSSSVPPYLFWYVQYPNQGLQLLLKYTTGATLVKGINGFEAEFKKSETSFHLTKPSAHMSDAAEYFCAVSENSGGSNYKLTFGKGTLLTVNPN

SEQ ID NO: 127 - protein, 5 aa, Homo sapiens
MNHEY

SEQ ID NO: 128 - protein, 6 aa, Homo sapiens
SMNVEV

SEQ ID NO: 129 - protein, 14 aa, Homo sapiens
CASSPGLNTGELFF

SEQ ID NO: 130 - protein, 132 aa, Homo sapiens
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSPGLNTGELFFGEGSRLTVL

SEQ ID NO: 131 - protein, 5 aa, Homo sapiens
MGHAR

SEQ ID NO: 132 - protein, 6 aa, Homo sapiens
YSYEKL

SEQ ID NO: 133 - protein, 15 aa, Homo sapiens
CASSQGTSGFYEQYF

SEQ ID NO: 134 - protein, 133 aa, Homo sapiens
MGCRLLCCAVLCLLGAVPIDTEVTQTPKHLVMGMTNKKSLKCEQHMGHRAMYWYKQKAKKPPELMFVYSYEKLSINESVPSRFSPECPNSSLLNLHLHALQPEDSALYLCASSQGTSGFYEQYFGPGTRLTVT

SEQ ID NO: 135 - protein, 5 aa, Homo sapiens
MNHEY

SEQ ID NO: 136 - protein, 6 aa, Homo sapiens
SMNVEV

SEQ ID NO: 137 - protein, 13 aa, Homo sapiens
CASSLSNYNEQFF

SEQ ID NO: 138 - protein, 131 aa, Homo sapiens
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSLSNYNEQFFGPGTRLTVL

SEQ ID NO: 139 - protein, 7 aa, Homo sapiens
NIATNDY

SEQ ID NO: 140 - protein, 5 aa, Homo sapiens
GYKTK

SEQ ID NO: 141 - protein, 16 aa, Homo sapiens
CLVGAGGNNAGNMLTF

SEQ ID NO: 142 - protein, 131 aa, Homo sapiens
MRQVARVIVFLTLSTLSLAKTTQPISMDSYEGQEVNITCSHNNIATNDYITWYQQFPSQGPRFIIQGYKTKVTNEVASLFIPADRKSSTLSLPRVSLSDTAVYYCLVGAGGNNAGNMLTFGGGTRLMVKPH

SEQ ID NO: 143 - protein, 6 aa, Homo sapiens
DSASNY

SEQ ID NO: 144 - protein, 7 aa, Homo sapiens
IRSNVGE

SEQ ID NO: 145 - protein, 14 aa, Homo sapiens
CAASMKTSYDKVIF

SEQ ID NO: 146 - protein, 133 aa, Homo sapiens
MTSIRAVFIFLWLQLDLVNGENVEQHPSTLSVQEGDSAVIKCTYSDSASNYFPWYKQELGKGPQLIIDIRSNVGEKKDQRIAVTLNKTAKHFSLHITETQPEDSAVYFCAASMKTSYDKVIFGPGTSLSVIPN

SEQ ID NO: 147 - protein, 6 aa, Homo sapiens
SSNFYA

SEQ ID NO: 148 - protein, 7 aa, Homo sapiens
MTLNGDE

SEQ ID NO: 149 - protein, 12 aa, Homo sapiens
CAPIYNTDKLIF

SEQ ID NO: 150 - protein, 134 aa, Homo sapiens
MEKNPLAAPLLILWFHLDCVSSILNVEQSPQSLHVQEGDSTNFTCSFPSSNFYALHWYRWETAKSPEALFVMTLNGDEKKKGRISATLNTKEGYSYLYIKGSQPEDSATYLCAPIYNTDKLIFGTGTRLQVFPN

SEQ ID NO: 151 - protein, 7 aa, Homo sapiens
TSESNYY

SEQ ID NO: 152 - protein, 8 aa, Homo sapiens
QEAYKQQN

SEQ ID NO: 153 - protein, 15 aa, Homo sapiens
CAFMTDTGANSKLTF

SEQ ID NO: 154 - protein, 147 aa, Homo sapiens
MGTLQGSAVSMTRVSLLWAVVVSTCLESGMAQTVTQSQPEMSVQEAETVTLSCTYDTSESNYYLFWYKQPPSRQMILVIRQEAYKQQNATENRFSVNFQKAAKSFSLKISDSQLGDTAMYFCAFMTDTGANSKLTFGKGITLSVRPD

SEQ ID NO: 155 - protein, 5 aa, Homo sapiens
SGHNS

SEQ ID NO: 156 - protein, 6 aa, Homo sapiens
FNNNVP

SEQ ID NO: 157 - protein, 14 aa, Homo sapiens
CASSLGGGSYEQYF

SEQ ID NO: 158 - protein, 133 aa, Homo sapiens
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSLGGGSYEQYFGPGTRLTVT

SEQ ID NO: 159 - protein, 5 aa, Homo sapiens
KAHSY

SEQ ID NO: 160 - protein, 6 aa, Homo sapiens
FQNEEL

SEQ ID NO: 161 - protein, 16 aa, Homo sapiens
CASSKTGTANTGELFF

SEQ ID NO: 162 - protein, 96 aa, Homo sapiens
MDCVPIKAHSYVYWYRKKLEEELKFLVYFQNEELIQKAEIINERFLAQCSKNSSCTLEIQSTESGDTALYFCASSKTGTANTGELFFGEGSRLTVL

SEQ ID NO: 163 - protein, 9 aa, Homo sapiens
KLVVVGVGV